(19) United States
(12) Patent Application Publication
(10) Pub. No.: US 2007/0197860 A1
(43) Pub. Date: Aug. 23, 2007

(54) Endoscope inserting section with case, and method for removing endoscope inserting section

(75) Inventor: Ryosuke Ishizaki, Tokyo (JP)

Correspondence Address: Thomas Spinelli;Scully, Scott, Murphy & Presser, Suite 300, 400 Garden City Plaza, Garden City, NY, 11530, US

(73) Assignee: OLYMPUS MEDICAL SYSTEMS CORP., Tokyo (JP)

(21) Appl. No.: 11/650,669

(22) Filed: Jan. 8, 2007

(30) Foreign Application Priority Data
Jan. 13, 2006 (JP) 2006-006795

(51) Int. Cl. A61B 1/00

(52) U.S. Cl. 600/101

(57) ABSTRACT

An endoscope inserting section with a case comprises: an inserting section including a flexible inserting section main body to be inserted into a body cavity, having a distal end rigid section at its distal end part, and a rotatable barrel body which is rotatably fitted on the inserting section main body and has a helical part with a helical convex-concave structure; and a housing case, including, an inserting section housing case main body for housing the inserting section, and an insertion assisting tool through which the inserting section is inserted to be guided into a body cavity. The insertion assisting tool retracts the inserting section to a position where an image of the distal end part of the insertion assisting tool can be picked up, when the inserting section is retracted.

This application claims benefit of Japanese Application No. 2006-6795 filed on Jan. 13, 2006, the contents of which are incorporated by this reference.

### BACKGROUND OF THE INVENTION

1. Field of the Invention

The present invention relates to an endoscope inserting section with a case which is used in a rotatable self-propelling endoscope, and a method for removing an endoscope inserting section.

2. Description of the Related Art

As is well known, endoscopes are extensively used in various fields including the medical field and the industrial field with a purpose to observe a region such as an inside of a cavity which cannot be directly viewed, and are generally configured to have an elongated inserting section to be inserted to a region to be examined.

Such endoscopes are known to have various structures. For example, a rotatable self-propelling endoscope which has an inserting section to be inserted into the large intestine per anum is known, the endoscope having a rotatable barrel body provided on the outer circumferential surface of the inserting section which has a helical-surface shaped part and is rotatable about an axis, and a rotation of the rotatable barrel body by a motor or the like causes a friction between the helical-surface shaped part and the intestinal wall, so that the inserting section can be automatically inserted into the large intestine and removed from the large intestine by the screwing action.

This technology in which a medical tool such as an endoscope is inserted into a body cavity by using friction between a rotatable member and tissues in a body cavity is for example disclosed in Japanese Patent Laid-Open No. 10-113396. In the Japanese Patent Laid-Open No. 10-113396, an advancing device of a medical apparatus which can guide the medical apparatus into a deep part of a living body cavity easily in a less invasive way is disclosed.

The advancing device is provided with a rotatable member having a rib which is disposed at an oblique angle with respect to an axial direction of the rotatable member. So, when the rotatable member rotates, the rotating power is converted into an impelling force by the rib, and thereby the impelling force causes the medical equipment coupled to the advancing device to be moved into a deeper part of body cavity.

Various types of endoscopes use the above technology. One example is a rotatable self-propelling endoscope device to be inserted into the large intestine per anum which has an inserting section having a flexible rotatable barrel body which is rotatable about an axis, and a rotation of the rotatable barrel body allows the endoscope to be automatically inserted into a body cavity. The rotatable barrel body is long enough to be inserted into a body cavity, and is formed of a metal which has a high rotation transmittance.

### SUMMARY OF THE INVENTION

An endoscope inserting section with a case, which is one embodiment of the present invention, comprises: an inserting section including an inserting section main body which is flexible to be inserted into a body cavity and has a distal end section provided with a distal end rigid section having an image pickup section thereto, and a rotatable barrel body with a helical-surface shaped part which is rotatably fitted onto the inserting section main body and has a helical-surface shaped part with a helical convex-concave structure; and a housing case which has an inserting section housing case main body for housing the inserting section main body and an insertion assisting tool through which the inserting section derived from the housing case main body inserted to be guided into the body cavity, the insertion assisting tool being configured to cause the inserting section to be retracted to a position where an image of the distal end part of the insertion assisting tool can be picked up when the inserting section is retracted.

A method for removing an endoscope inserting section is another embodiment of the present invention and is a method for removing an inserting section which has a flexible inserting section main body, a rotatable barrel body which is rotatably fitted on the inserting section main body to allow the inserting section main body to be self-propelled through a body cavity, a distal end rigid section with an image pickup section, and a bending section, from the body cavity, comprising: rotating the rotatable barrel body so that the inserting section is retracted, and housing a predetermined part of the inserting section into an inner bore of an insertion assisting tool which is inserted in the body cavity, stopping the rotation of the rotatable barrel body, and removing the inserting section from the body cavity with the insertion assisting tool.

According to the above present invention, when an image of the distal end part of the insertion assisting tool is displayed in an endoscope image, a user can recognize that the distal end part of the inserting section is located at a removal end position where a region can be observed, and then stop the rotation of the inserting section to remove the inserting section.

The above and other objects, features and advantages of the invention will become more clearly understood from the following description referring to the accompanying drawings.

### BRIEF DESCRIPTION OF THE DRAWINGS

FIG. 1 is a diagram showing an entire structure of a first embodiment of a rotatable self-propelling endoscope system according to the present invention;

FIG. 2 is a cross sectional diagram showing a part of a distal end section, a bending section, an inserting section main body, and a rotatable barrel body of an inserting section according to the first embodiment of an endoscope;

FIG. 3 is a perspective diagram showing an insertion assisting tool of the first embodiment;

FIG. 4 is an exploded perspective diagram showing an insertion assisting tool to which a guide tube is connected according to the first embodiment;

FIG. 5 is a cross sectional diagram showing an insertion assisting tool having a guide tube connected thereto according to the first embodiment;

FIG. 6 is a cross sectional diagram showing a proximal end part of an insertion assisting tool having a guide tube connected thereto according to the first embodiment;

FIG. 7 is a cross sectional diagram showing an insertion assisting tool taken along a line VII-VII of FIG. 6 according to the first embodiment;

FIG. 8 is a cross sectional diagram showing a distal end part of an insertion assisting tool into which an inserting section is inserted according to the first embodiment;

FIG. 9 is an exploded perspective diagram showing a housing case main body according to the first embodiment;

FIG. 10 is a cross sectional diagram showing a housing case main body according to the first embodiment;

FIG. 11 is a plan diagram showing a housing case main body seen from one side according to the first embodiment;

FIG. 12 is an enlarged diagram showing one side of a housing case main body to which a guide tube fixing member is mounted according to the first embodiment;

FIG. 13 is an enlarged plan diagram showing the housing case main body of FIG. 12 with a guide tube fixing member mounted to one side thereof according to the first embodiment;

FIG. 14 is an exploded perspective diagram showing a guide tube fixing member to which an impelling force generating member is provided according to the first embodiment;

FIG. 15 is a fragmentary cross sectional diagram showing a guide tube fixing member to which an impelling force generating member is provided, seen from a vertical direction of a housing case main body according to the first embodiment;

FIG. 16 is a fragmentary cross sectional diagram showing a guide tube fixing member to which an impelling force generating member is provided, seen from a horizontal direction of a housing case main body according to the first embodiment;

FIG. 17 is a plan diagram showing an impelling force generating member according to the first embodiment;

FIG. 18 is a cross sectional diagram illustrating an action of an impelling force generating member in a guide tube fixing member, with a rotatable barrel body being inserted therethrough, according to the first embodiment;

FIG. 19 is a plan diagram showing a modified impelling force generating member according to the first embodiment;

FIG. 20 is cross sectional diagram illustrating an action of the impelling force generating member of FIG. 19, with a rotatable barrel body being inserted therethrough, according to the first embodiment;

FIG. 21 is a fragmentary cross sectional diagram showing a guide tube fixing member toward an operating section according to the first embodiment;

FIG. 22 is across sectional diagram showing a connection between a guide tube toward an operating section and a connector cover according to the first embodiment;

FIG. 23 is a cross sectional diagram showing a part of a connector cover to which a guide tube toward an operating section is connected according to the first embodiment;

FIG. 24 is a cross sectional diagram showing a cross section of a connector box taken along a line XXIV-XXIV of FIG. 23 according to the first embodiment;

FIG. 25 is a cross sectional diagram showing a rotatable barrel body of an inserting section which is inserted into a guide tube according to the first embodiment;

FIG. 26 is a cross sectional diagram showing a rotatable barrel body of an inserting section in a housing case main body according to the first embodiment;

FIG. 27 is a cross sectional diagram of a modified example of the insertion assisting tool, showing a distal end part of the insertion assisting tool to which an impelling force generating member is provided according to the first embodiment;

FIG. 28 is a diagram illustrating a method for removing an inserting section, and showing an action of an insertion assisting tool inserted into the rectum per anus of a patient according to the first embodiment;

FIG. 29 a diagram illustrating an action of an inserting section which is inserted into the large intestine, having reached the sigmoid colon according to the first embodiment;

FIG. 30 is a diagram illustrating an action of an inserting section which is inserted into the large intestine, having reached near the cecum;

FIG. 31 is a diagram illustrating an action of an inserting section having reached a removal end position near the rectum according to the first embodiment;

FIG. 32 is a diagram showing an example of a monitor display when an inserting section has reached the removal end position shown in FIG. 31 according to the first embodiment;

FIG. 33 is a perspective diagram showing a structure of an insertion assisting tool through which an inserting section is inserted according to a second embodiment of the present invention;

FIG. 34 is a cross sectional diagram showing the insertion assisting tool of FIG. 33 according to the second embodiment;

FIG. 35 is a diagram showing an example of a monitor display when an inserting section has reached the removal end position shown in FIG. 34 according to the second embodiment;

FIG. 36 is a perspective diagram showing a modified example of the cylinder-shaped member of the second embodiment, and shows a structure of an insertion assisting tool through which an inserting section is inserted;

FIG. 37 is a cross sectional diagram showing a structure of an insertion section which is inserted through an insertion assisting tool according to a third embodiment of the present invention;

FIG. 38 is a block diagram showing an electrical structure of the control device 3 which is electrically connected to an inserting section main body according to the third embodiment;

FIG. 39 is a block diagram showing a structure of a main component circuit block for controlling a retraction of an inserting section according to a fourth embodiment of the present invention;

FIG. 40 is a diagram showing an insertion assisting tool which is inserted through an inserting section, and a structure of a main component circuit block in a controlling device which is electrically connected to the insertion assisting tool according to a fifth embodiment of the present invention;

FIG. 41 is a top view showing an inserting section with a case seen from the top thereof according to a sixth embodiment of the present invention; and

FIG. 42 is a cross sectional diagram showing a distal end part of an insertion assisting tool to which an impelling force generating member is provided according to a seventh embodiment of the present invention.

### DETAILED DESCRIPTION OF THE PREFERRED EMBODIMENTS

Now, several embodiments of the present invention will be explained below with reference to the drawings.

### First Embodiment

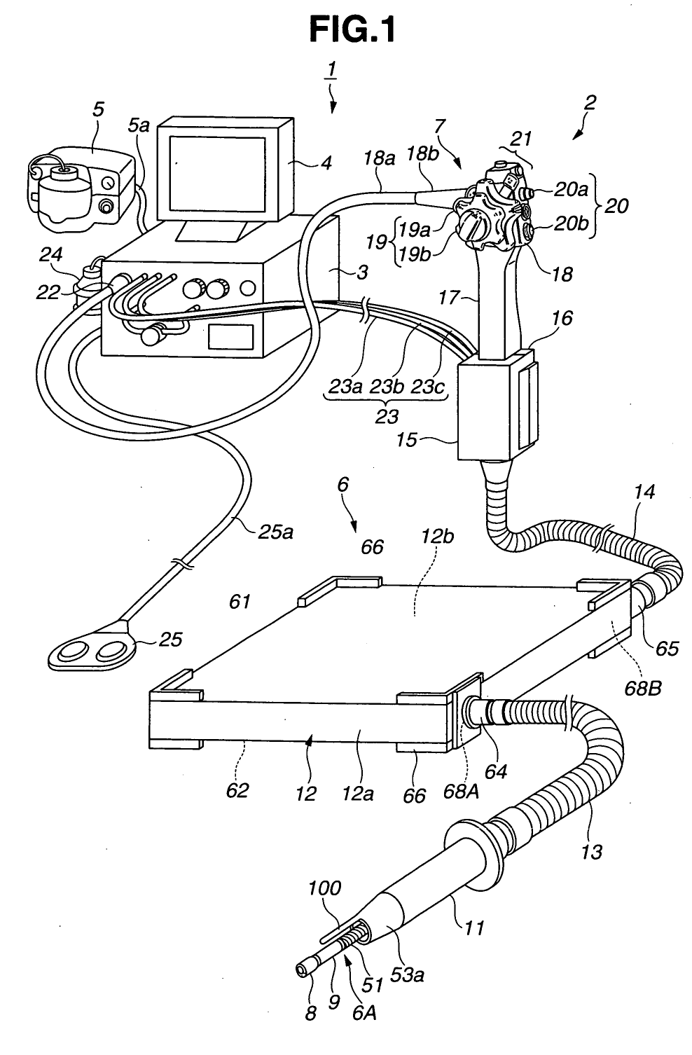

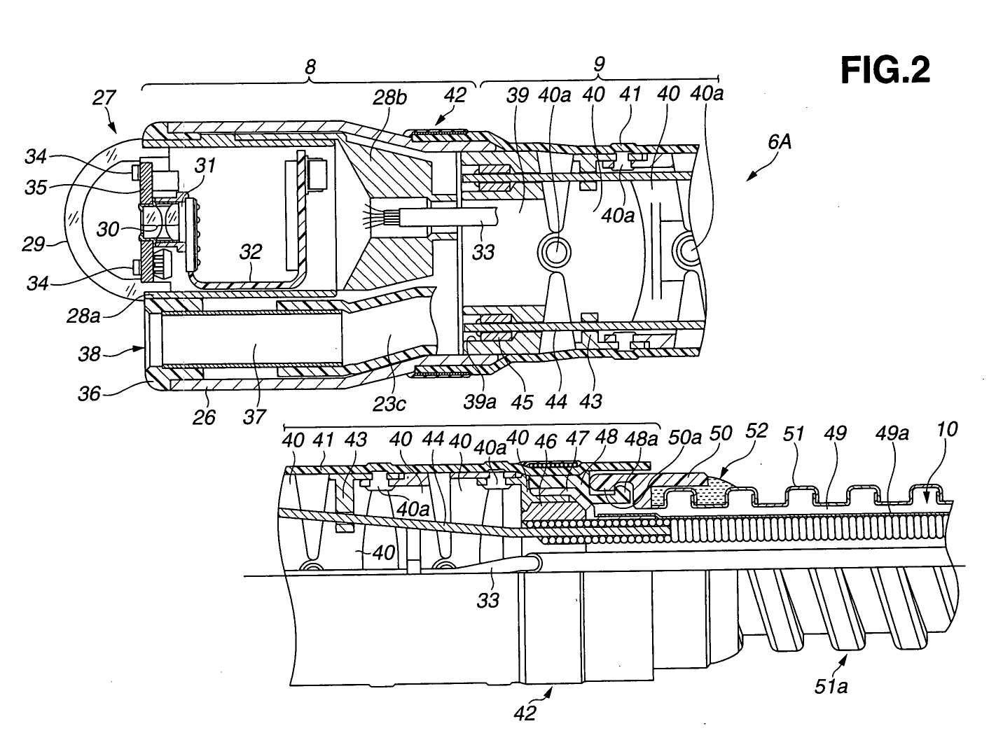

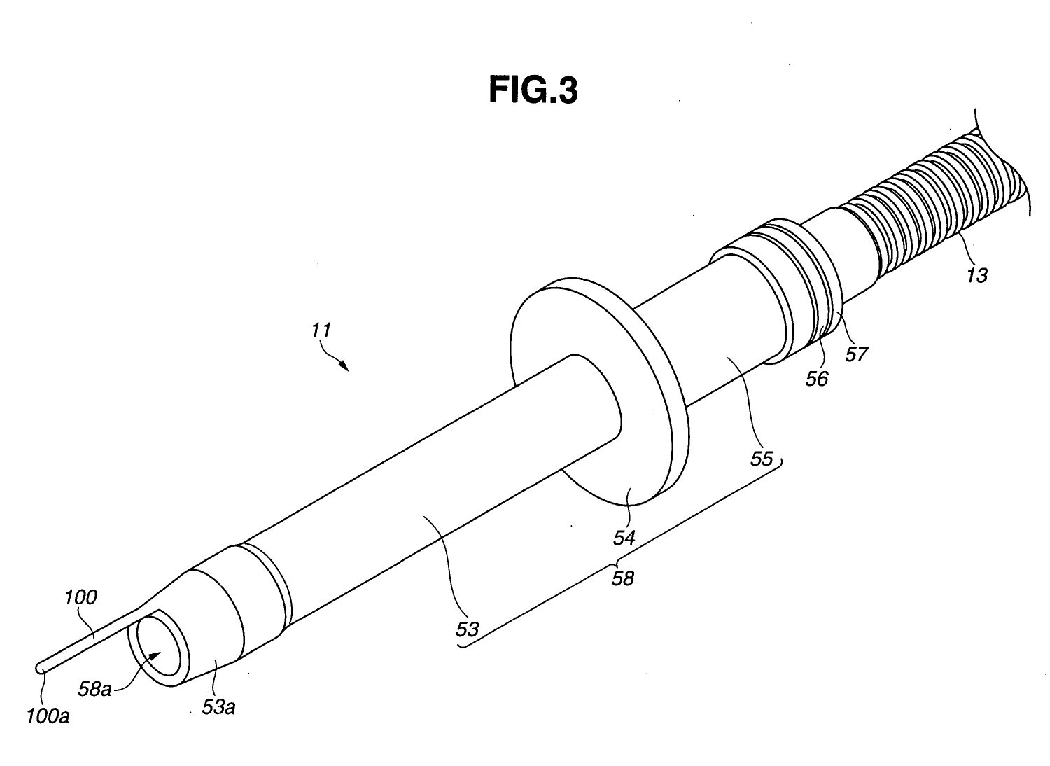

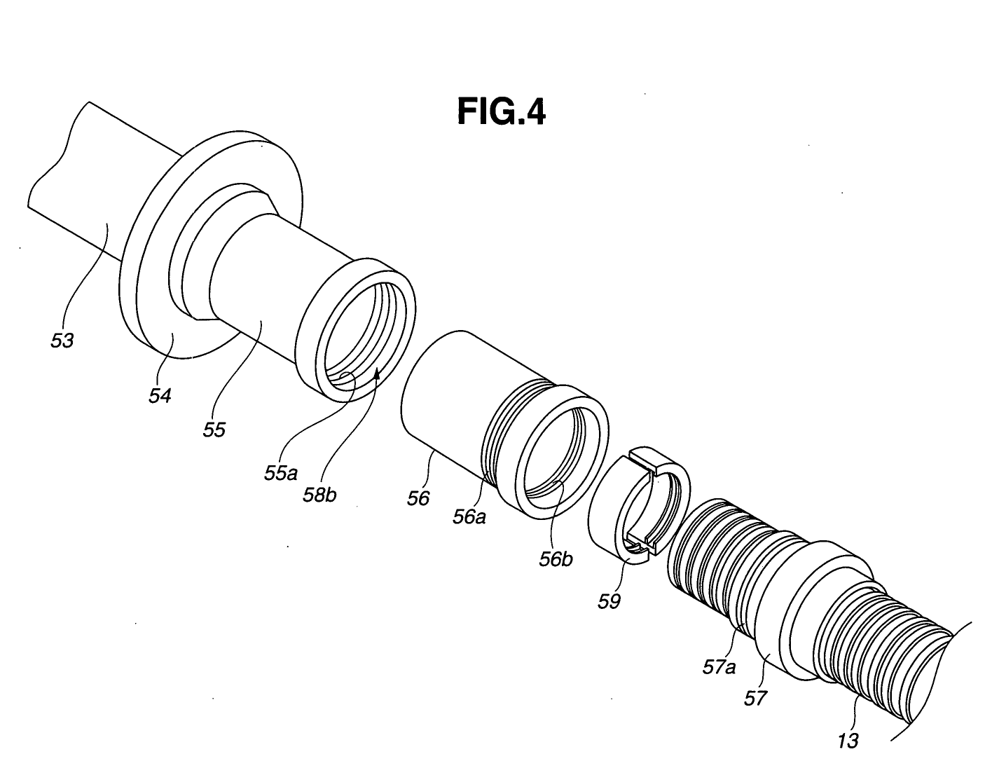

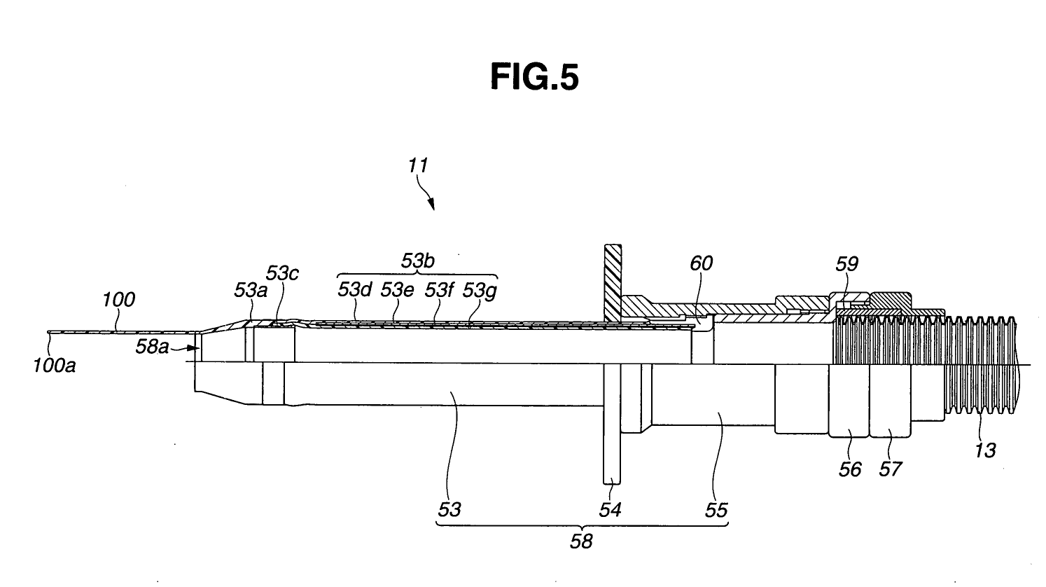

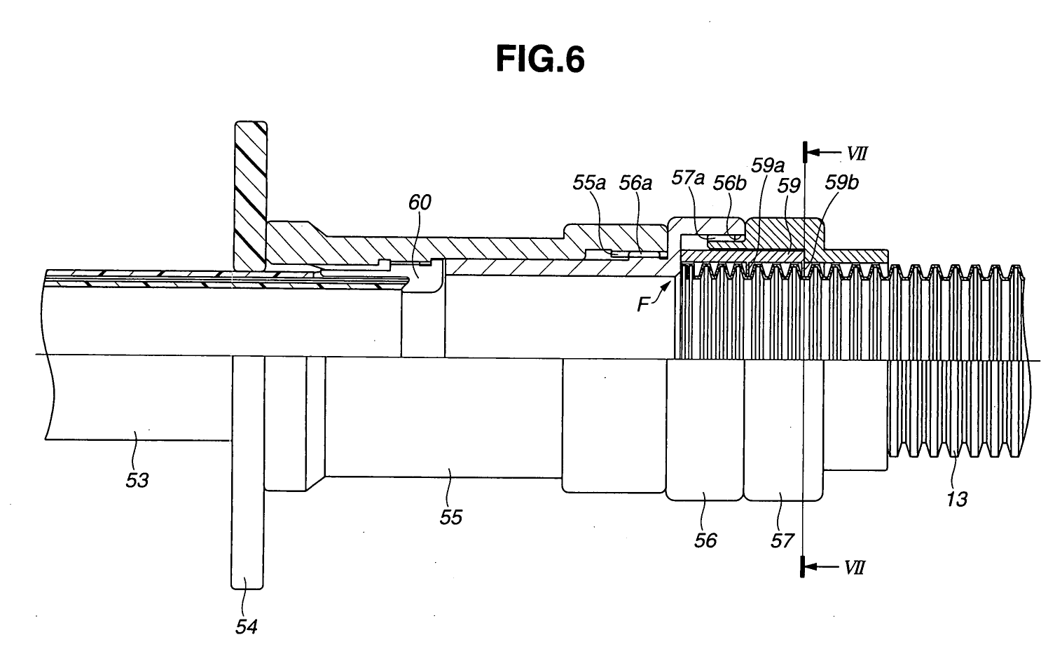

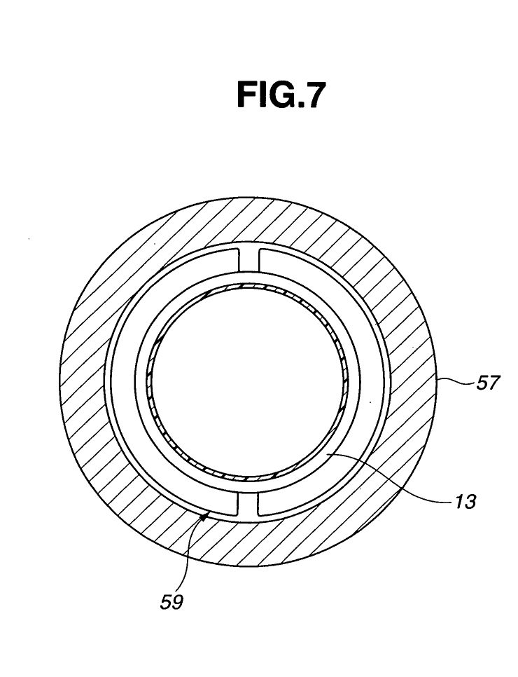

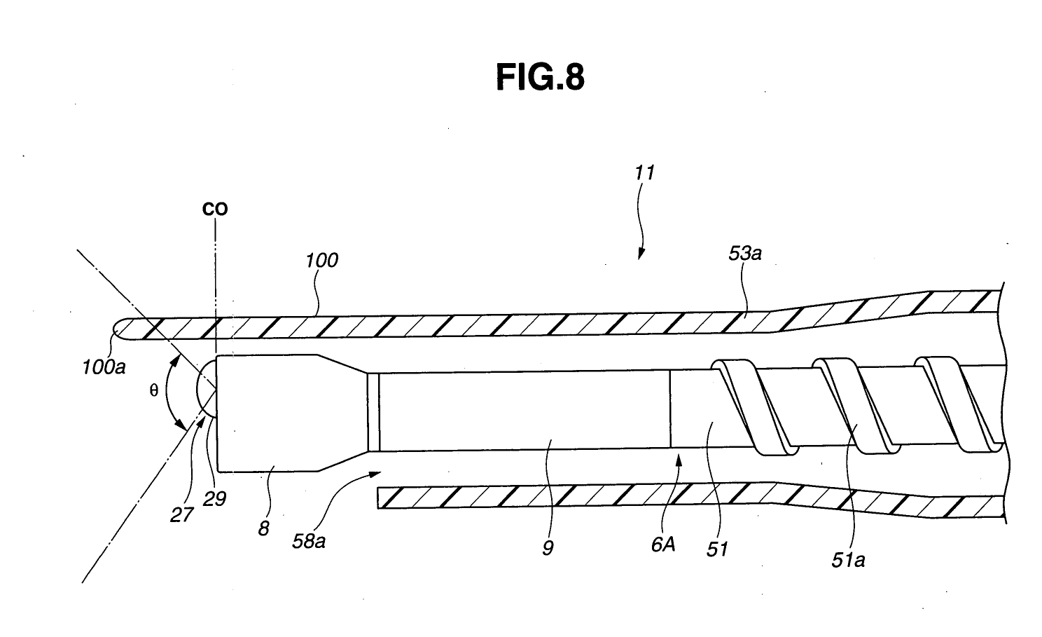

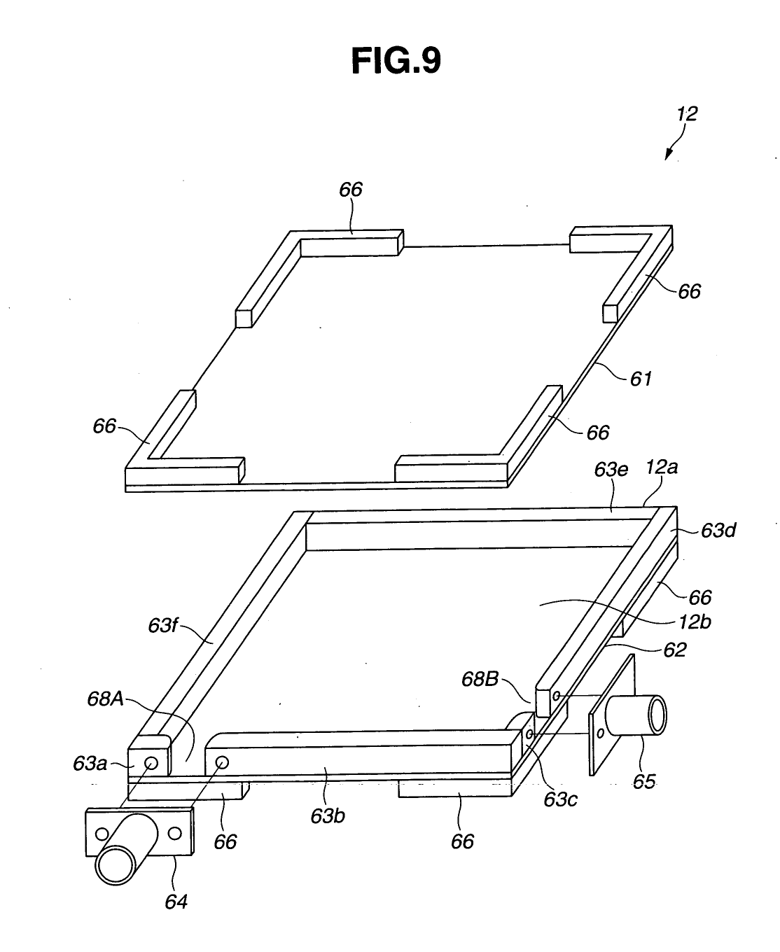

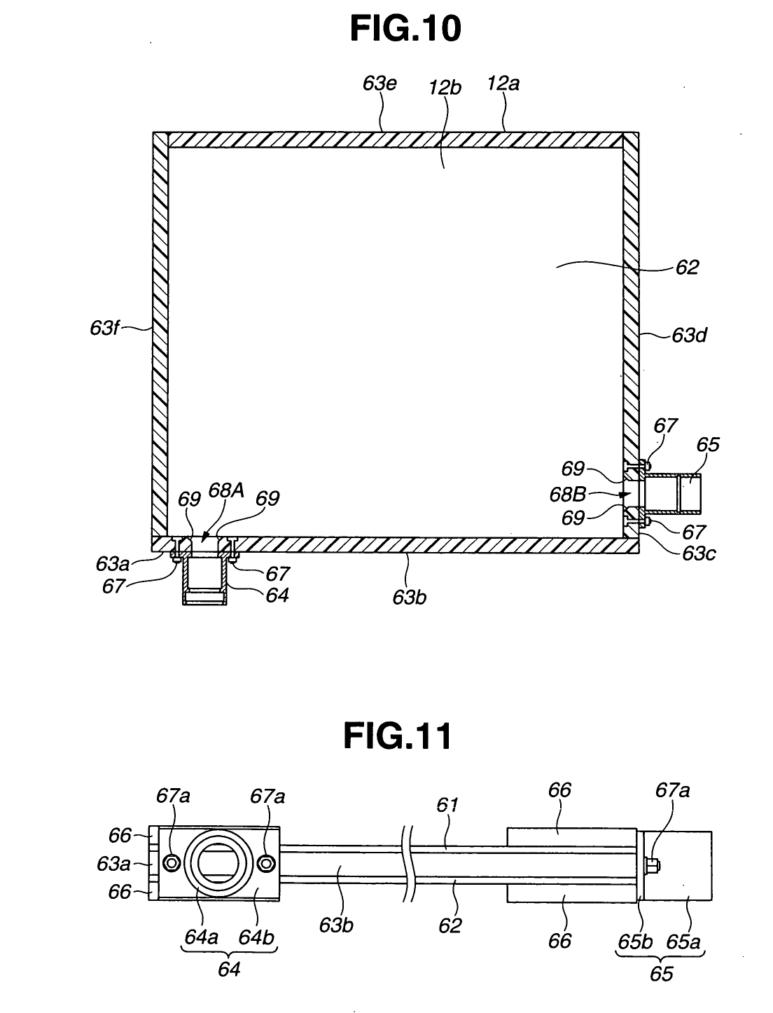

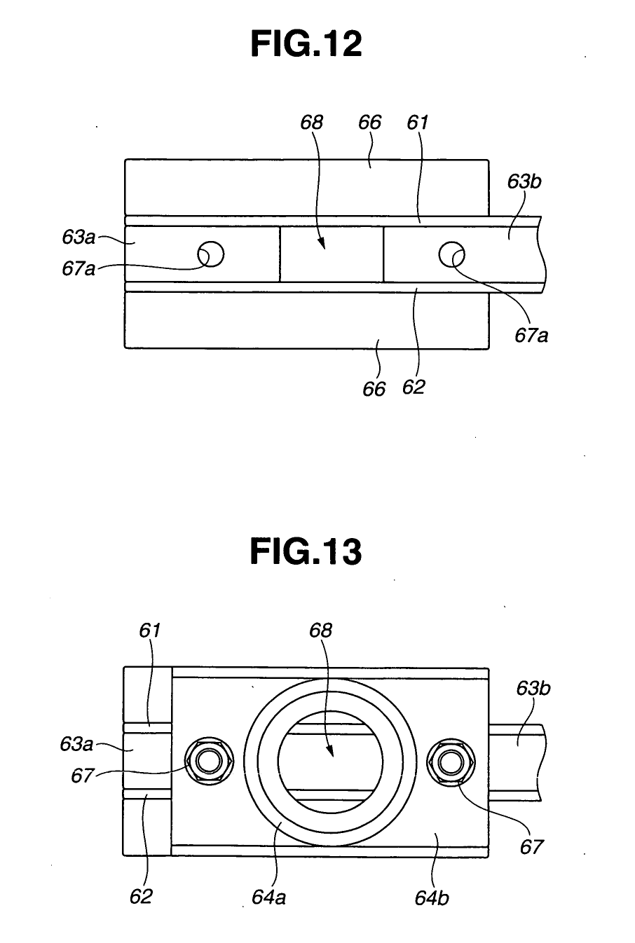

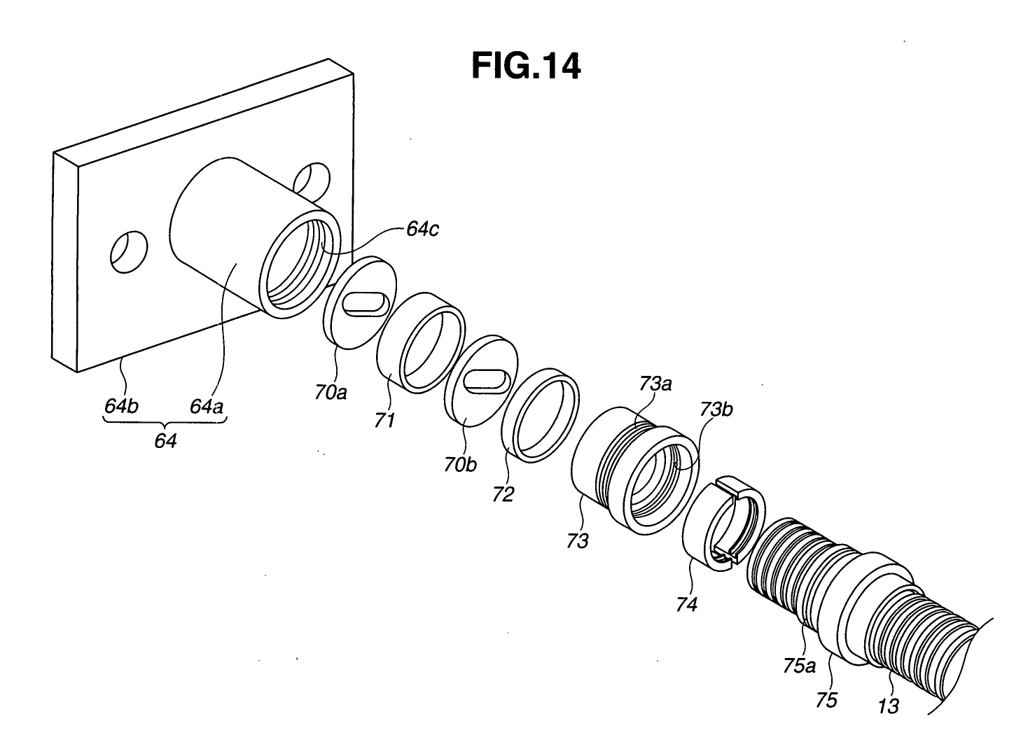

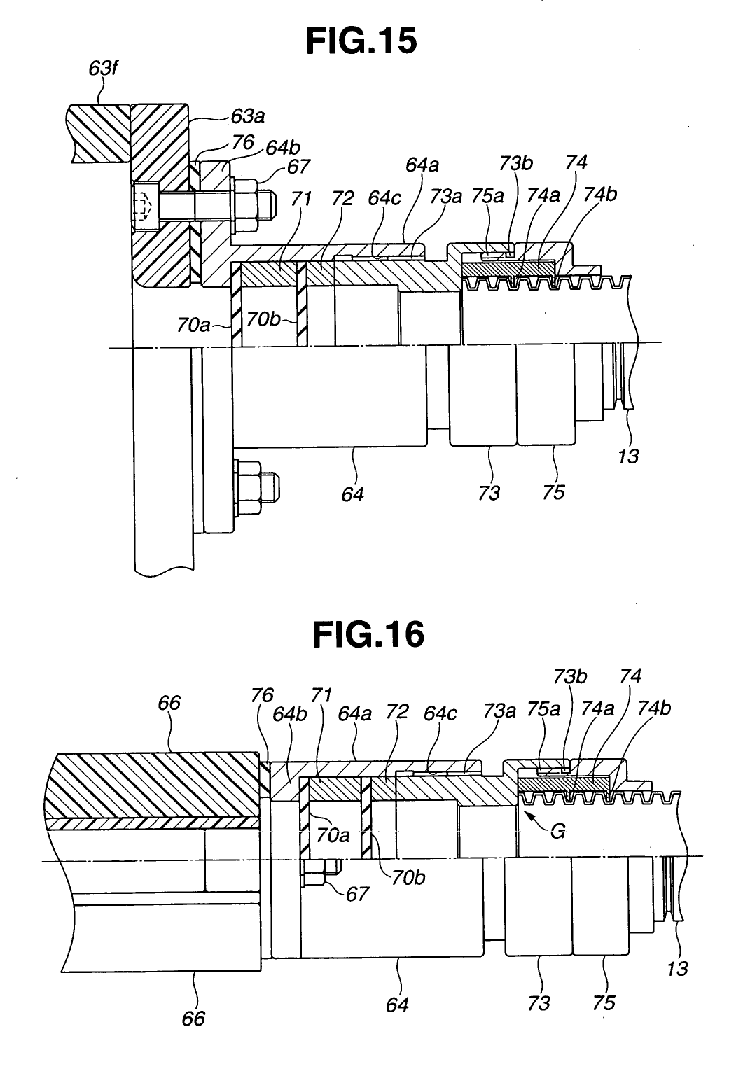

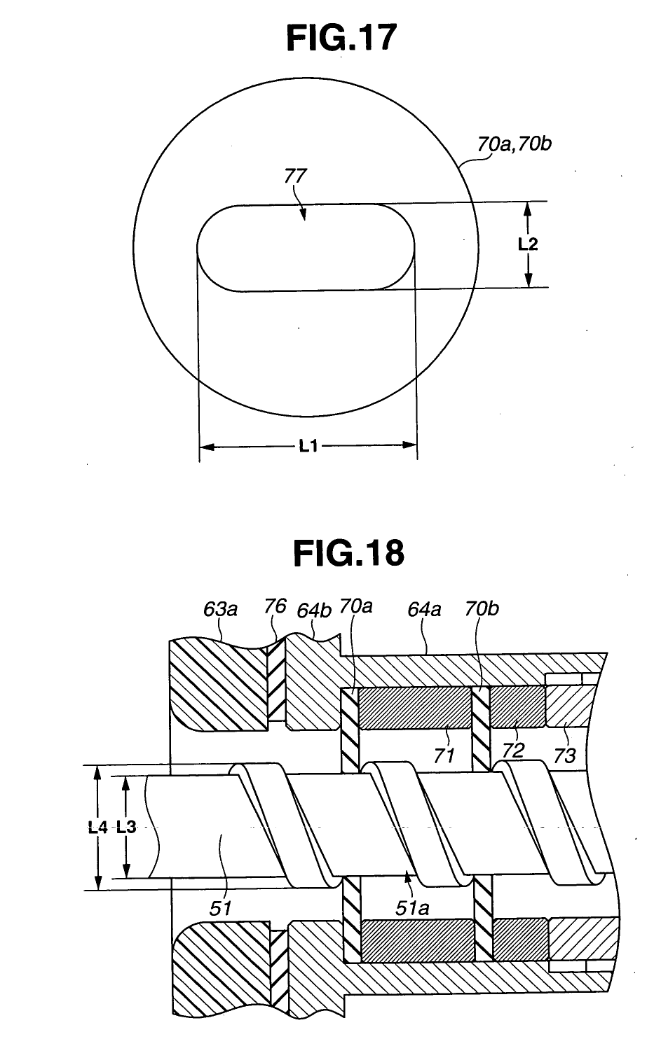

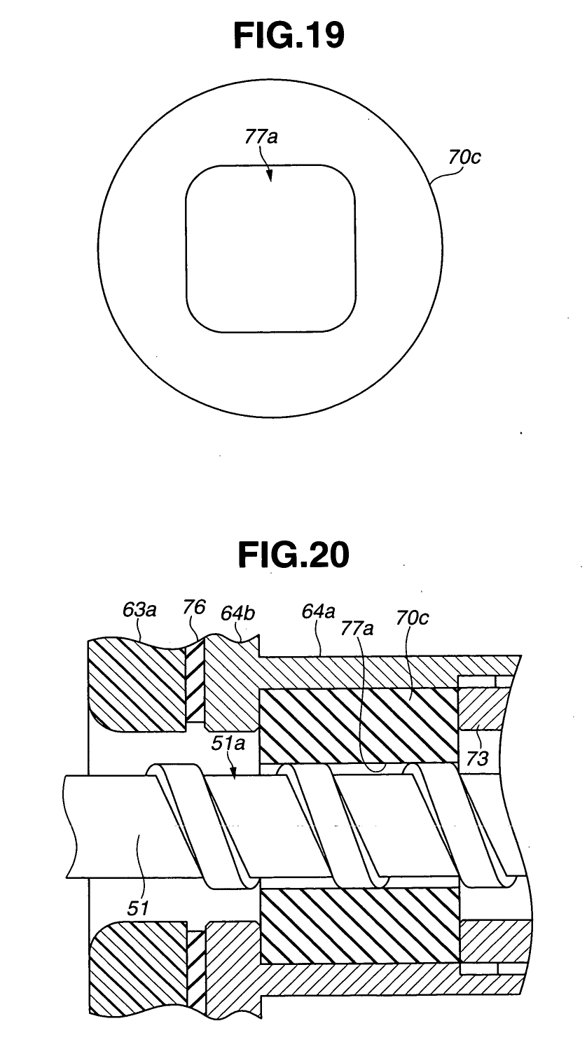

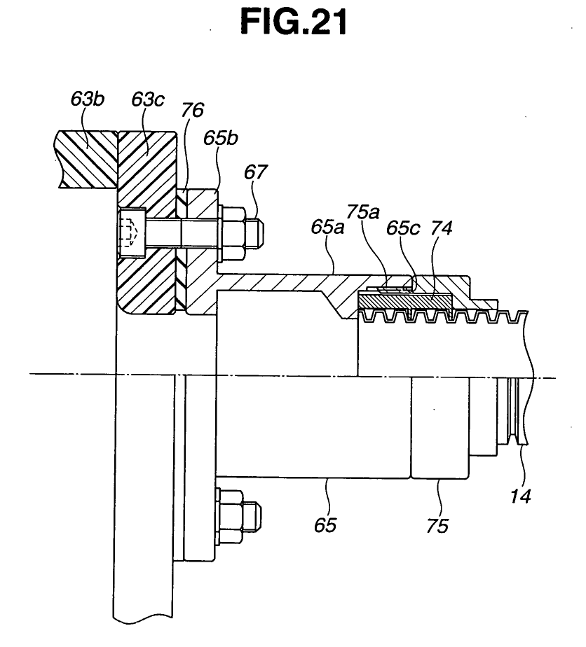

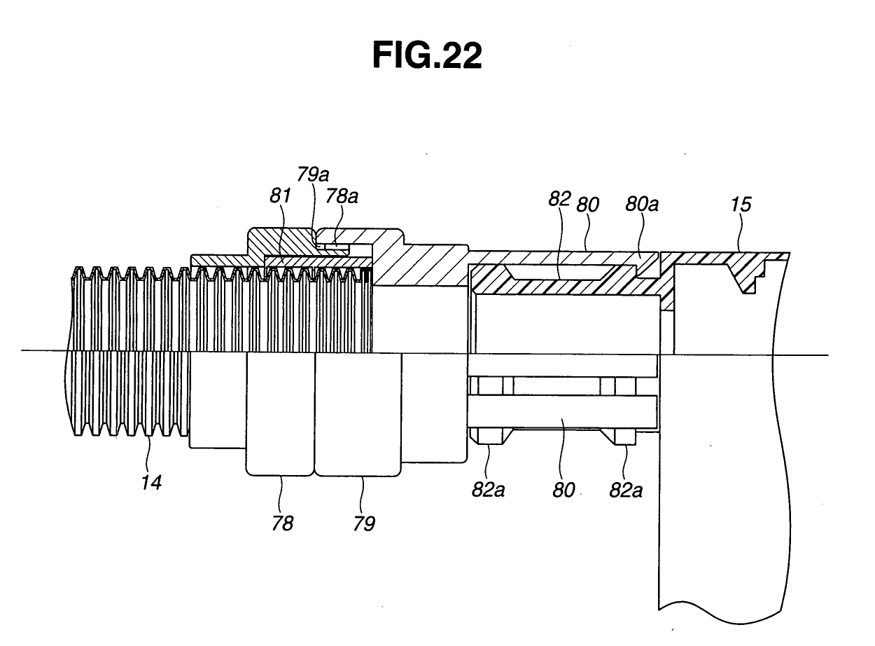

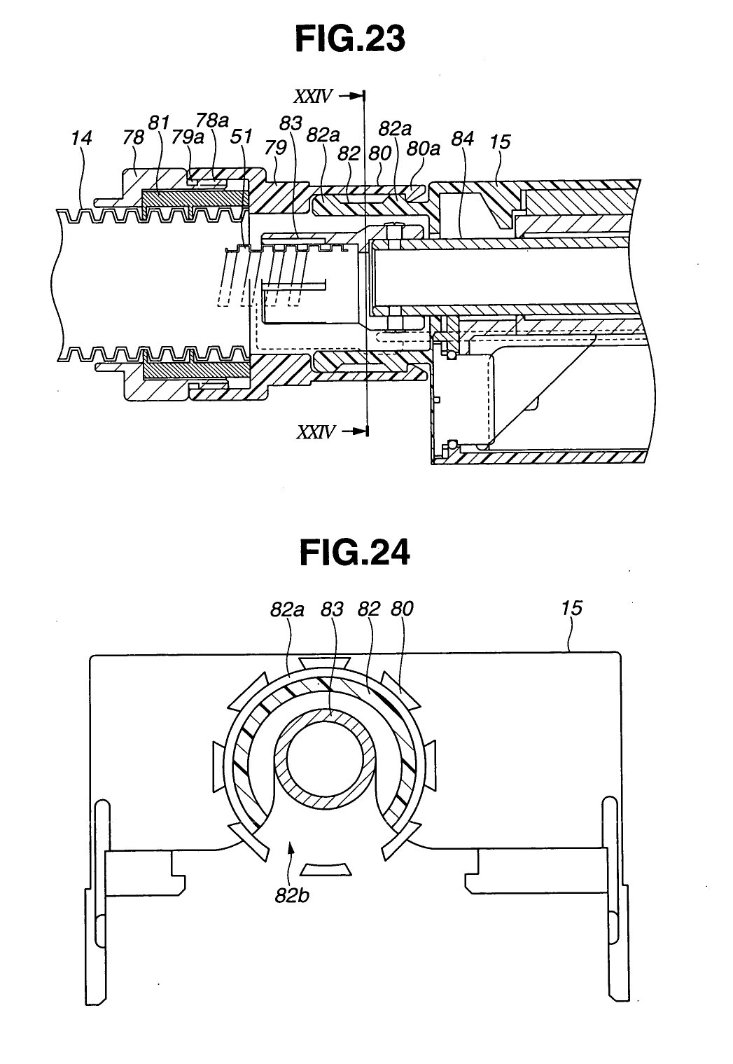

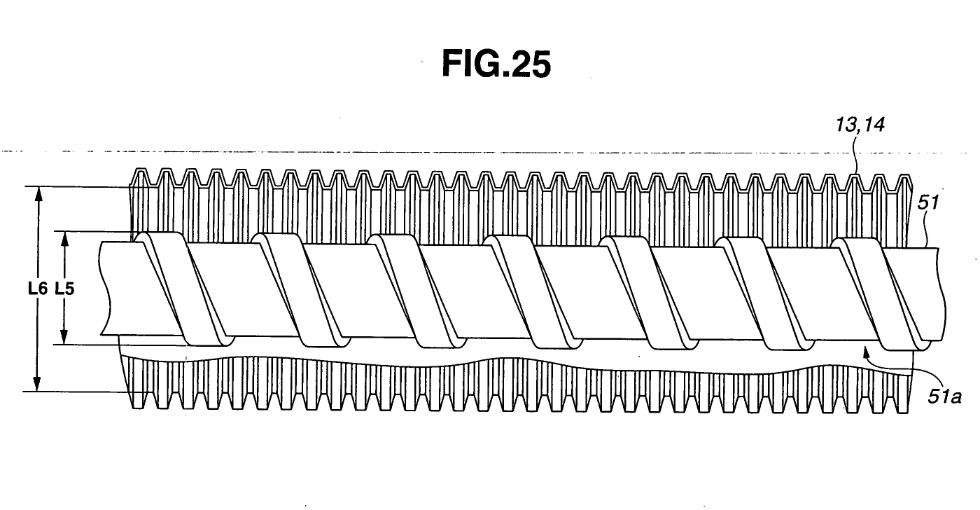

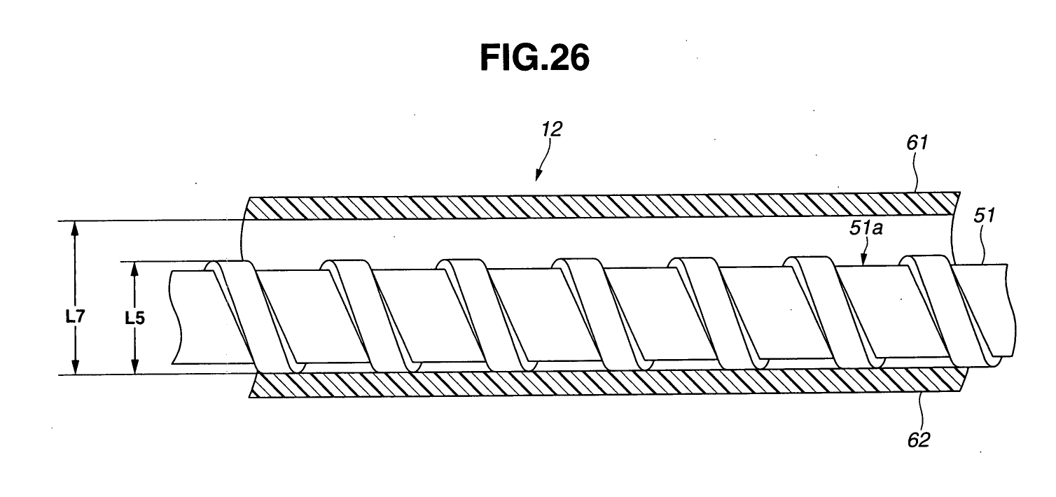

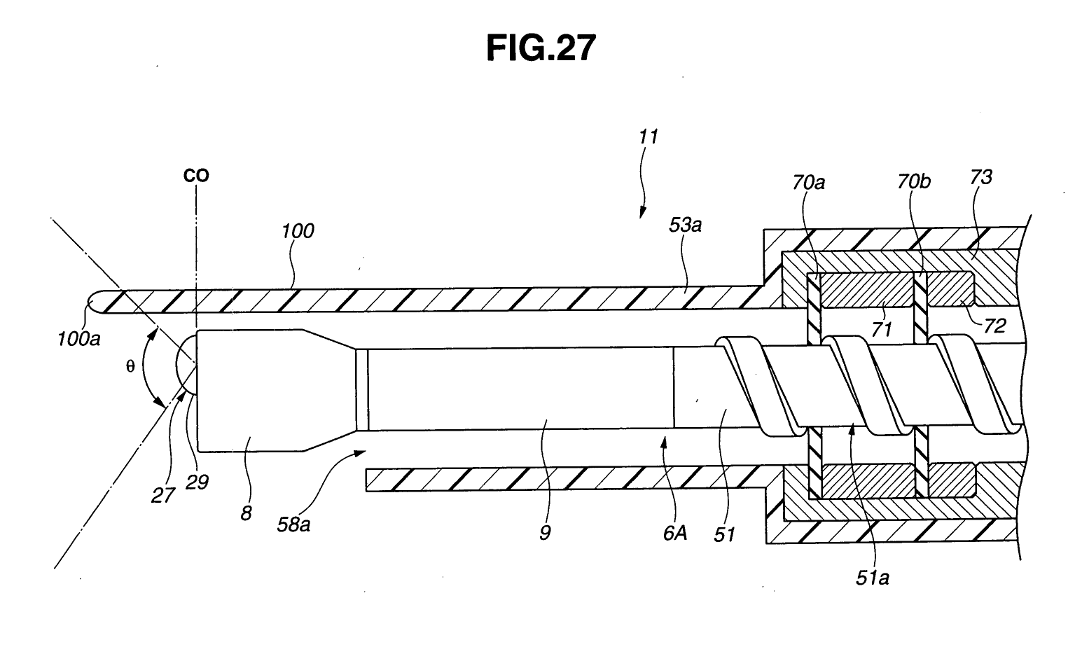

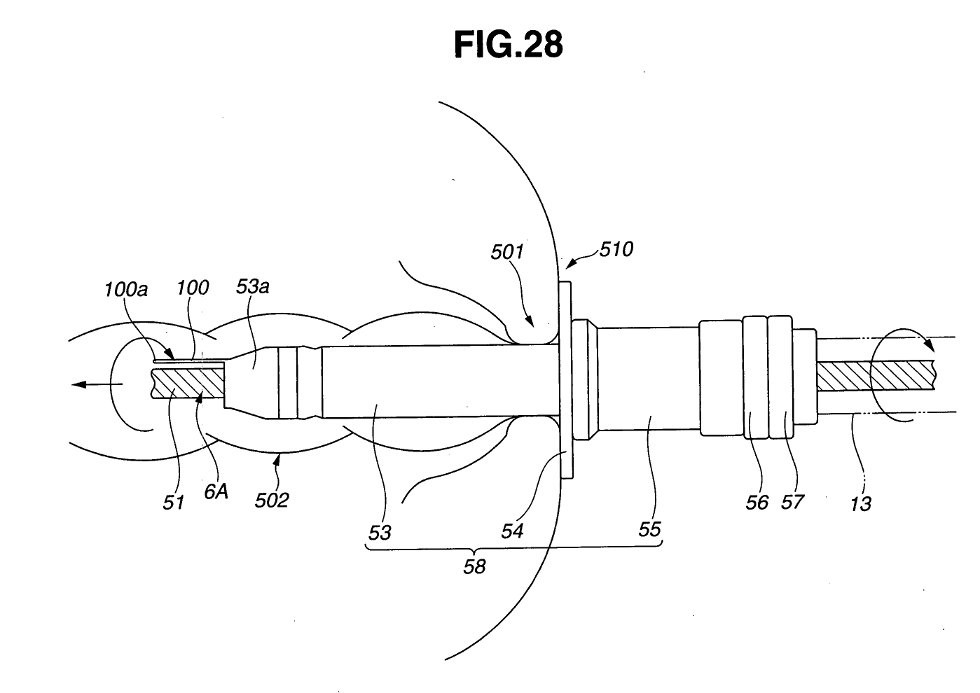

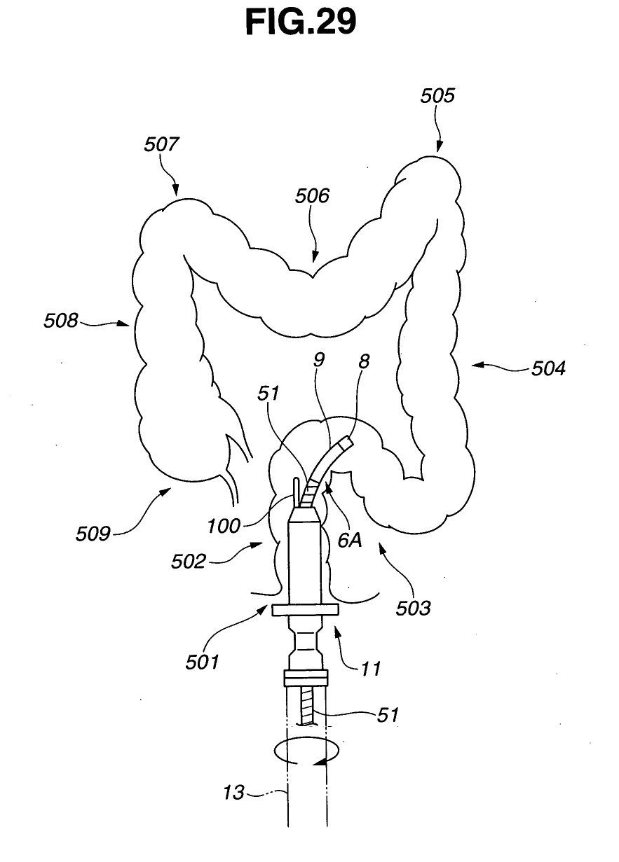

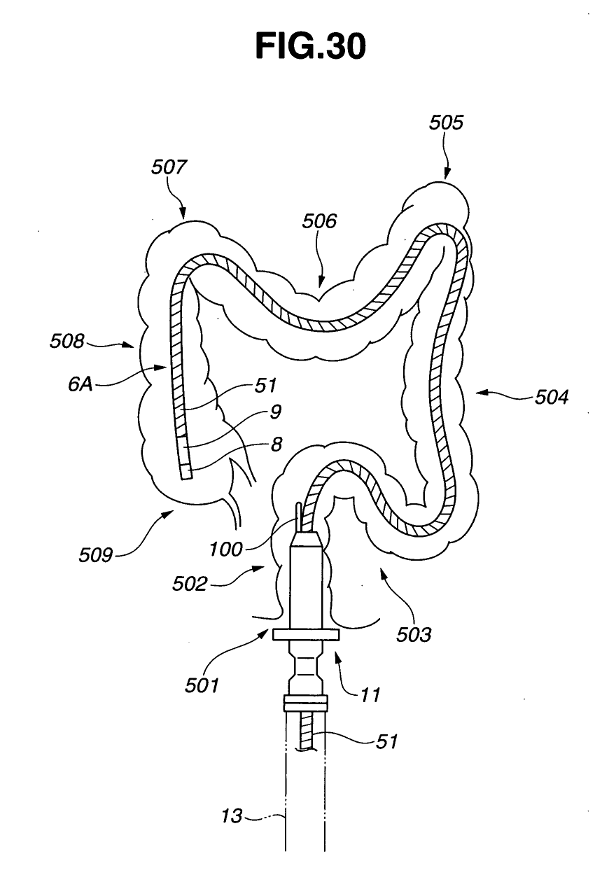

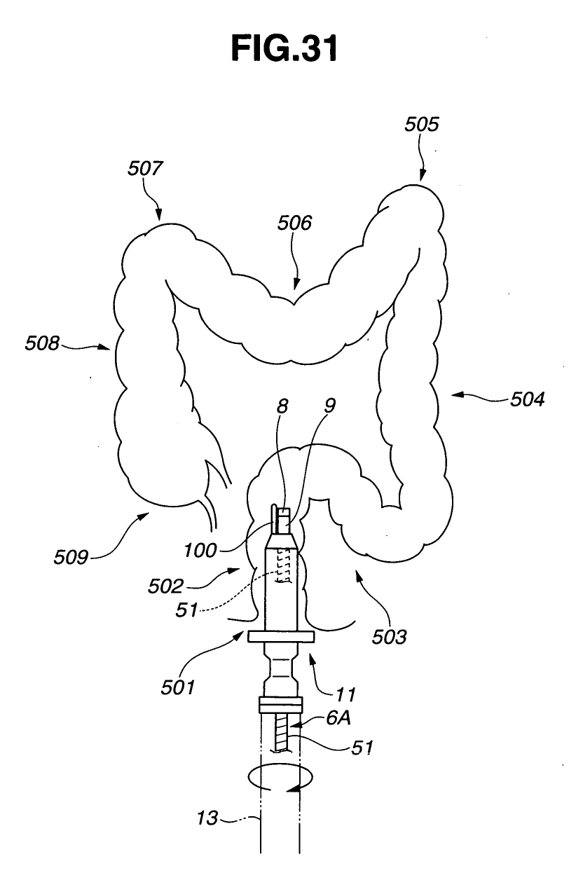

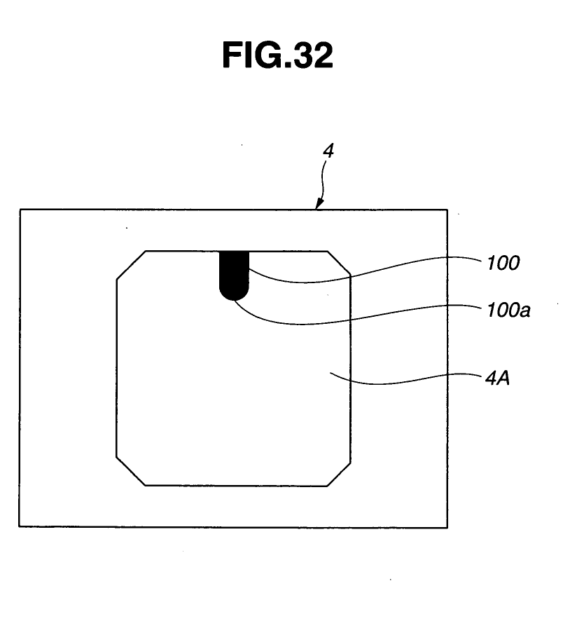

FIG. 1 to FIG. 32 are diagrams showing a first embodiment of the present invention: FIG. 1 is a diagram showing an entire structure of a rotatable self-propelling endoscope system; FIG. 2 is a cross sectional diagram showing a part of a distal end section, a bending section, and a rotatable barrel body of an endoscope; FIG. 3 is a perspective diagram showing an insertion assisting tool; FIG. 4 is an exploded perspective diagram showing an insertion assisting tool to which a guide tube is connected; FIG. 5 is a cross sectional diagram showing an insertion assisting tool having a guide tube connected thereto; FIG. 6 is a cross sectional diagram showing a proximal end part of an insertion assisting tool having a guide tube connected thereto; FIG. 7 is a cross sectional diagram showing an insertion assisting tool taken along a line VII-VII of FIG. 6; FIG. 8 is a cross sectional diagram showing a distal end part of an insertion assisting tool into which an inserting section is inserted; FIG. 9 is an exploded perspective diagram showing a housing case main body; FIG. 10 is a cross sectional diagram showing a housing case main body; FIG. 11 is a plan diagram showing a housing case main body seen from one side; FIG. 12 is an enlarged diagram showing one side of a housing case main body to which a guide tube fixing member is mounted; FIG. 13 is an enlarged plan diagram showing the housing case main body of FIG. 12 with a guide tube fixing member mounted to one side thereof; FIG. 14 is an exploded perspective diagram showing a guide tube fixing member to which an impelling force generating member is provided; FIG. 15 is a fragmentary cross sectional diagram showing a guide tube fixing member to which an impelling force generating member is provided, seen from a vertical direction of a housing case main body; FIG. 16 is a fragmentary cross sectional diagram showing a guide tube fixing member to which an impelling force generating member is provided, seen from a horizontal direction of a housing case main body; FIG. 17 is a plan diagram showing an impelling force generating member; FIG. 18 is a cross sectional diagram illustrating an action of an impelling force generating member in a guide tube fixing member, with a rotatable barrel body being inserted therethrough; FIG. 19 is a plan diagram showing a modified impelling force generating member; FIG. 20 is cross sectional diagram illustrating an action of the impelling force generating member of FIG. 19, with a rotatable barrel body being inserted therethrough; FIG. 21 is a fragmentary cross sectional diagram showing a guide tube fixing member toward an operating section; FIG. 22 is a cross sectional diagram showing a connection between a guide tube toward the operating section and a connector cover; FIG. 23 is a cross sectional diagram showing a part of a connector cover to which a guide tube toward an operating section is connected; FIG. 24 is a cross sectional diagram showing a cross section of a connector box taken along a line XXIV-XXIV of FIG. 23; FIG. 25 is a cross sectional diagram showing a rotatable barrel body of an inserting section which is inserted into a guide tube; FIG. 26 is a cross sectional diagram showing a rotatable barrel body of an inserting section in a housing case main body; and FIG. 27 is a cross sectional diagram of a modified insertion assisting tool, showing a distal end part of the insertion assisting tool to which an impelling force generating member is provided. FIG. 28 to FIG. 32 are diagrams illustrating a method for removing an inserting section of the first embodiment: FIG. 28 is a diagram showing an action of an insertion assisting tool which is inserted into the rectum per anus of a patient; FIG. 29 a diagram illustrating an action of an inserting section which is inserted into the large intestine and reached to the sigmoid colon; FIG. 30 is a diagram illustrating an action of an inserting section which is inserted into the large intestine and reached near the cecum; FIG. 31 is a diagram illustrating an action of an inserting section which is at a removal end position near the rectum; and FIG. 32 is a diagram showing an example of a monitor display when an inserting section reaches at the removal end position shown in FIG. 31.

First, with reference to FIG. 1, an entire structure of a rotatable self-propelling endoscope system 1 will be explained below.

As shown in FIG. 1, a rotatable self-propelling endoscope system (hereinafter, simply referred to as an endoscope system) 1, which can be used with a method for removing endoscope inserting section with a case according to the present invention, includes a rotatable self-propelling endoscope (hereinafter, simply referred to as an endoscope) 2, a control device 3, a monitor 4, and an aspirator 5.

The endoscope 2 has an endoscope inserting section with a case (hereinafter, simply referred to as an inserting section with a case) 6, and an operating section 7.

The inserting section with a case 6 has an inserting section 6A, and an inserting section housing case main body (hereinafter, simply referred to as a housing case main body) 12.

The inserting section with a case 6 is configured to have, in order from the distal end, a distal end rigid section (hereinafter, simply referred to as a distal end section) 8 which constitutes the inserting section 6A, a bending section 9, a rotatable barrel body 51 having an inserting section main body 10 inside thereof, an insertion assisting tool 11 which constitutes the housing case main body 12, a housing case main body 12, a guide tube toward the distal end 13 which is a corrugated tube interposed between the insertion assisting tool 11 and the housing case main body 12, a guide tube toward the operating section 14 which is a corrugated tube interposed between the operating section 7 and the housing case main body 12, and a connector cover 15 to which an end of the guide tube toward the operating section 14 is connected.

The inserting section with a case 6 constitutes the endoscope inserting section of the present invention. The inserting section with a case 6 is also configured to be removably attached to the operating section 7 so that the inserting section 6A can perform predetermined functions.

The operating section 7 has a motor box 16 to which a connector cover 15, constructing one element of the inserting section with a case 6, is removably attached, a holding section 17, and a main operating section 18.

The main operation secting 18 is provided with a bending knob 19 for bending the bending section 9 of the endoscope inserting section with a case 6 in four directions (to the upper, lower, right and left directions with respect to an endoscope image captured by the endoscope 2), buttons 20 for delivering or sucking fluids, and switches 21 for controlling various optical systems such as am image pickup system or an illuminating system.

The bending knob 19 is provided to one surface of the main operating section 18 of the operating section 7 in a way that the two generally disc-shaped knobs are superimposed. The two knobs are rotatably arranged, and include a U(UP)/D(DOWN) bending knob 19a which is provided on a side toward the main operating section 18 for operations in the upper and lower directions with respect to the bending section 9, and an R (RIGHT)/L (LEFT) bending knob 19b which is provided on the U/D bending knob 19a for operations in the right and left directions with respect to the bending section 9.

The operating section 18 has a side from which a universal cord 18a, that is an electrical cable, is extended. The operating section 18 also has an anti-bending section 18b at the root of the universal cord 18a.

The universal cord 18a has an extended end at which a connector section 22 is provided. The connector section 22 is connected to the control device 3.

The buttons 20 on the one side of the main operating section 18 include an air supply/water supply button 20a for supplying air or liquid from the distal end section 8 of the endoscope 2 into a subject body, and a suction button 20b for sucking liquids such as drainage in the subject body from the distal end section 8 of the endoscope 2.

The connector cover 15 which is removably attached to the motor box 16 has three tubes 23 which are inserted through the inserting section with a case 6 and extend from the connector cover 15. The three tubes 23 include an air supply tube 23a, a water supply tube 23b, and a suction tube 23c. The extended ends of the three tubes 23 are individually connected to a front surface of the control device 3 at predetermined positions via removable connectors.

The control device 3 is provided with a water supply tank 24. The water supply tank 24 stores sterilized water therein. A predetermined operation of the air supply/water supply button 20a of the main operating section 18 causes the sterilized water to be supplied to the water supply tube 23b by the control device 3, which is ejected from the distal end section 8 of the endoscope 2.

A predetermined operation of the air supply/water supply button 20a of the main operating section 18 causes air to be supplied to the air supply tube 23a from a compressor in the control device 3 (not shown), which is ejected from the distal end section 8 of the endoscope 2.

An operation of the suction button 20b causes drainage and the like to be sucked from the distal end section 8 of the endoscope 2, which is sent through the suction tube 23c and the control device 3 to the aspirator 5.

Although the rotatable self-propelling endoscope system 1 of this embodiment uses the aspirator 5, other suction systems applied to the hospital may be used.

The control device 3 is connected to a foot switch 25 via an electrical cable 25a. The foot switch 25 is used to rotate the inserting section 6A of the endoscope 2 in a predetermined direction and to stop the rotation. A switch for rotating the inserting section 6A and stopping the inserting section 6A is also provided to the main operating section 18 of the operating section 7, but is not shown.

The front surface of the control device 3 is provided with a power switch, a dial for changing a rotation speed of the inserting section 6A of the endoscope 2, and the like. The motor box 16 of the operating section 7 has a motor (not shown) built therein for rotating the inserting section 6A.

The control device 3 is electrically connected to the monitor 4. The monitor 4 displays an endoscope image captured by the endoscope 2.

Next, with reference to FIG. 2, the distal end section 8, the bending section 9, the inserting section main body 10, and the rotatable barrel body 51 which are a part of the inserting section 6A of the endoscope 2 will be explained below.

First, the distal end section 8 will be explained.

The distal end section 8 includes a rigid and generally circular ring-shaped main body ring 26 which includes a biocompatible synthetic resin, and an image pickup unit 27 which is one element of the image pickup section.

The image pickup unit 27 has an outer contour formed with a generally circular ring-shaped retaining ring 28a which includes a synthetic resin and is received in the main body ring 26, a generally circular ring-shaped cover ring 28b which includes a metal and is fitted on a proximal end of the retaining ring 28a, and a cover member 29 which is formed into a dome shape with a biocompatible clear synthetic resin and is fitted on a distal end opening of the retaining ring 28a to seal the opening air-tight.

The above members form a space in the image pickup unit 27, into which objective lenses 30, an image pickup device 31 such as a CCD or a CMOS which is disposed at a position where the image pickup light incident to the objective lenses 30 is focused, and a flexible printed circuit (FPC) 32 into which an image signal photoelectrically converted by the image pickup device 31 is input are provided.

The FPC 32 is connected with a communication cable 33. The communication cable 33 is inserted through the bending section 9 and the inserting section main body 10 to be connected to a connector (not shown) which is disposed in the connector cover 15 (see FIG. 1).

The objective lenses 30 are retained by a retaining ring which is fixed by a plate member 35, and the plate member 35 has a plurality of LEDs 34 which are provided to surround the objective lenses 30 as illuminating members. The plate member 35 is formed into a generally circle shape to be fixedly attached to an inner surface which is located on an extension of a generally center line of the cover member 29. The objective lenses 30 are disposed so that an optical axis passes through a generally center position of the plate surface of the plate member 35.

The image pickup unit 27 having a configuration as described above is disposed at an offset position relative to the center of the main body ring 26, and is fixed to the main body ring 26 by a distal end cap 36 which is provided in an opening at the distal end of the main body ring 26.

There is a gap between the retaining ring 28a and the main body ring 26 of the image pickup unit 27, into which a distal end part of the suction tube 23c and a suction tube 37 connected to the proximal end of the suction tube 23 are provided. The suction tube 37 has a distal end section to which the distal end cap 36 is fixedly attached.

The distal end cap 36 has an opening 38 formed therein for sucking. Not shown, but tube paths which are in communication with the air supply tube 23a and the water supply tube 23b are provided to utilize the space between the retaining ring 28a and the main body ring 26, and the tube paths have openings which are also formed in the distal end cap 36.

Next, the bending section 9 will be explained.

In the bending section 9, a rigid distal end bending piece 39 which is fitted on the proximal end opening of the main body ring 26 at the distal end section 8, and a plurality of rigid bending pieces 40 (which are also called as bending nodal rings) are pivotally arranged in an array about a pivot 40a. The pieces 39 and 40 are covered with a bendable outer covering 41 which includes a biocompatible elastic member such as fluorocarbon rubber. The outer covering 41 has a distal end section which is fixedly attached to the proximal end part of the main body ring 26 at the distal end section 8 by a spool bonding section 42.

The plurality of bending pieces 40 have wire guides 43 which protrude from the inner circumferential surface toward the center of the pieces 40. The wire guides 43 have bending wires 44 (which is also called as angle wires) inserted therethrough.

Four bending wires 44 are provided in the bending section 9, and the distal end parts of the bending wires 44 (only two of them are shown in FIG. 2) are individually welded with a cylindrical locking member 45 by using solder or the like. The locking members 45 of the bending wires 44 are locked to four locking holes 39a formed in the distal end bending piece 39, respectively.

The four locking holes 39a are formed at the positions in a plane, which is perpendicular to the axis of the distal end bending piece 39, separated by generally equal spaces to locate each hole at each quarter of the plane. The distal end bending piece 39 is oriented so that each locking hole 39a is positioned corresponding to the upper, lower, right, and left directions of the endoscope image. Thus, the four bending wires 44 are fixedly held at four points separated by generally equal spaces in the upper, lower, right, and left directions.

The four bending wires 44 are inserted through the inserting section main body 10 to be provided into the connector cover 15.

Each of the bending wires 44 has a wire joint (not shown) at the proximal end part thereof. The wire joints of bending wires 44 are coupled to coupling members (not shown) which are provided in the holding section 17 respectively, when the connector cover 15 is integral with the motor box 16.

Each of the coupling members is coupled by a bending mechanism (not shown) and a chain (not shown) which are provided in the main operating section 18 and work with the bending knob 19. In other words, a rotation of the bending knob 19 causes each of the coupling members to be alternately towed or loosened by the bending mechanism, which in turn causes each of the bending wires 44 to be alternately towed or loosened to work with the coupling members.

So, a plurality of bending pieces 40 are pivoted in response to the tow or loose of each of the four bending wires 44. This makes the bending section 9 bent in the above four directions.

The proximal end part of the bending section 9 is provided with: a first cap 46 which includes a metal for fixing a coil pipe and is fitted in the inside of the most distal end bending piece 40; a second cap 47 which includes a metal for fixing an inner layer tube and is fitted on the outer circumferential surface of the most distal end bending piece 40; and a third cap 48 which includes a synthetic resin for rotatably engaging the rotatable barrel body and is fitted on the outer circumferential surface of the second cap 47. The caps 46 to 48 are securely and fixedly attached to each other by using an adhesive or the like.

The above described bendable outer covering 41 is also fixedly attached to the third cap 48 by the spool bonding section 42.

The above described bending wires 44 are individually inserted through coil sheaths 49 from the first cap 46 at the proximal ends thereof. The coil sheaths 49 have distal end parts which are fixed to the first cap 46 by using solder or the like. The coil sheaths 49 used in this embodiment has an incompressible configuration with a wire which is closely wound into a pipe shape.

The second cap 47 has a proximal end part to which the distal end part of a flexible inner layer tube 49a is fixed, the flexible inner layer tube 49a being inserted through the inserting section main body 10. The inner layer tube 49a may be a tube body made of a thin wire by blading the wire into a cylindrical shape to make the tube body flexible.

The third cap 48 has a proximal end part to which a projection 48a is provided. The third cap 48 is completely covered with the bendable outer covering 41 to provide a gap between the outer circumferential surface of the projection 48a and the outer covering 41. The action of the projection 48a will be explained later.

Next, the inserting section main body 10 and the rotatable barrel body 51 of the inserting section 6A will be explained below.

The inserting section 6A includes the inserting section main body 10 and the rotatable barrel body 51.

The inserting section main body 10 is provided inside with the above described inner layer tube 49a, the four coil sheaths 49 through which each of the bending wires 44 is inserted, the communication cable 33, and various tubes 23 (not shown). That is, also as seen from FIG. 2, the inner layer tube 49a is disposed at the outer most position to protect other elements inside.

The rotatable barrel body 51 has a cap 50 which includes a synthetic resin at the distal end part thereof for coupling, and the distal end part and the resin being fixedly attached by an adhesive 52.

The cap 50 has a distal end part including an uneven part 50a which is engaged with the projection 48a of the third cap to effect a so-called snap-fit function. In other words, the cap 50 and the third cap 48 are individually rotatable about the axes of their own.

The rotatable barrel body 51 coupled to the cap 50 is a flexible cylindrical body formed by helically winding biocompatible metal plate members which are processed to have a convex-concave cross section. The rotatable barrel body 51 is made by engaging the above described convex-concave plate members with almost no gap therebetween to provide a helical-surface shaped part 51a to the outer circumferential surface of the rotatable barrel body 51, which is a helical convex part (or a helical concave part, or a convex part which protrudes to be arranged in an array along a helix).

Specifically, the rotatable barrel body 51 is a helical tube which is made, in consideration of its insertion into a body cavity, of stainless steel for example, to have a predetermined diameter size. The rotatable barrel body 51 may have various pitches of the convex and concave parts, a helical angle, and the like, by changing the size of the convex and concave parts of the plate member.

The rotatable barrel body 51 is configured to be rotatable about an axis in the direction in which the rotatable barrel body 51 is inserted. And a rotation of the rotatable barrel body 51 causes the helical-surface shaped part 51a of the outer circumferential surface to contact an inner wall of a body cavity in a subject to generate a thrust which makes the rotatable barrel body 51 move by itself in the direction in which the rotatable barrel body 51 is inserted.

At this point, the cap 50 which is fixedly attached to the distal end part of the rotatable barrel body 51 contacts the third cap 48 at the proximal end part of the bending section 9 to press the bending section 9, which generates an impelling force for advancing the entire inserting section 6A including the distal end section 8 into a deeper part of the body cavity.

The rotatable barrel body 51 is configured to receive a rotation driving force from a motor (not shown) which is provided in the motor box 16 of the operating section 7 (see FIG. 1).

In this embodiment, the rotation driving force from a motor (not shown) is transmitted to the proximal end of the rotatable barrel body 51 to rotate the rotatable barrel body 51, but the configuration of the rotatable barrel body 51 is not limited to this. For example, the rotatable barrel body 51 may be configured so that the rotation driving force from a motor (not shown) is transmitted to the midst of the rotatable barrel body 51 to rotate the entire rotatable barrel body 51, or is transmitted to the distal end part of the rotatable barrel body to rotate the rotatable barrel body 51.

Next, with reference to FIG. 3 to FIG. 8, the insertion assisting tool 11 will be explained below.

The insertion assisting tool 11 of this embodiment makes the inserting section 6A derived from the housing case main body 12 inserted therethrough to guide the inserting section 6A into a body cavity, and is configured to allow the inserting section 6A to be retracted to a position where an image of the distal end part of the insertion assisting tool 11 can be picked up.

The insertion assisting tool 11 may be configured to allow the inserting section 6A to be retracted to a position where an image of the inner circumferential surface of the insertion assisting tool 11 can be picked up.

Specifically, as shown in FIG. 3, the insertion assisting tool 11 includes an insertion tube 53 with a projecting part 100 which forms a projection at the distal end part of the insertion assisting tool 11, a deviation preventing section 54 which is means for preventing deviation, a retaining tube 55, a first securing ring 56, and a second securing ring 57.

The insertion tube 53, the deviation preventing section 54, and the retaining tube 55 form a tube main body 58 of the insertion assisting tool 11 as a unit.

The retaining tube 55 is a metal ring formed into a generally cylindrical shape (which may be a rigid cylindrical body which includes a synthetic resin, plastic, and the like), and has projected outer circumferential parts in the direction of the outer diameter at the both ends thereof. The retaining tube 55 includes a proximal end having an inner circumferential surface in which a female screw part 55a is formed as shown in FIG. 4.

As shown in FIG. 4, the first securing ring 56 is a metal ring formed into a generally cylindrical shape (which may be a rigid cylindrical body which includes a synthetic resin, plastic, and the like), and has a projected outer circumferential part in the direction of the outer diameter at the proximal end thereof. The first securing ring 56 includes a proximal end having an outer circumferential surface in which a male screw part 56a is formed and an inner circumferential surface in which a female screw part 56b is formed.

The second securing ring 57 is a metal ring formed into a generally cylindrical shape (which may be a rigid cylindrical body which includes a synthetic resin, plastic, and the like), and has projected outer circumferential parts in the direction of the outer diameter at the midst part thereof. The second securing ring 57 includes a distal end having an outer circumferential surface on which a male screw part 57a is formed.

Into the second securing ring 57, a guide tube toward the distal end 13 is provided to be inserted. The guide tube toward the distal end 13 is inserted through the second securing ring 57 with the distal end thereof being protruded from the second securing ring 57, and a clamp ring 59 which is separated into two parts is fitted on the outer circumferential surface of the protruded distal end part. The guide tube toward the distal end 13, and the fastening of the insertion assisting tool 11 by using the clamp ring 59 will be explained later.

As shown in FIG. 5, the insertion tube 53 includes a generally circular ring-shaped distal end insertion tube 53a which includes a flexible synthetic resin such as silicon and has a tapered outer circumferential surface to have a tapered shape, an inserting barrel body 53b which forms the main body of the insertion tube 53, and a joining ring 53c which couples the distal end insertion tube 53a and the inserting barrel body 53b at the inner circumferential surfaces thereof.

The distal end insertion tube 53a has a part of the outer circumferential surface at the distal end section to which a projecting part 100 is provided, the projecting part 100 projecting in the direction of an insertion axis of the insertion tube 53. The projecting part 100 is formed into a cylindrical shape for example by using a flexible synthetic resin such as silicon as in the case of the distal end insertion tube 53a. The projecting part 100 has a distal end part which is formed in an arc shape not to give any damage to body tissues.

The projecting part 100 is formed to have a length in the insertion direction thereof, as described later, of a preset size, depending on a removal end position of the inserting section 6A which is to be inserted and a viewing angle θ of the image pickup unit 27 at the distal end section 8.

In this embodiment, the projecting part 100 of a cylindrical shape as shown in FIG. 3 to FIG. 5 has been described, but the shape of the projecting part 100 is not limited to this. The projecting part 100 may be extended from the entire or a part of the outer circumference of the distal end part of the distal end insertion tube 53a to be formed into a barrel-like shape or a generally barrel-like shape which partly has a notch.

The inserting barrel body 53b includes, in order from the outermost surface thereof, an outer tube 53d which is formed of a synthetic resin such as polyurethane, a blade 53e which is formed into a barrel-like shape by weaving metal wires into a mesh, a flex tube 53f which is a metallic helix tube, and an inner tube 53g which is formed of a synthetic resin such as polyurethane.

The outer tube 53d, the blade 53e, the flex tube 53f, and the inner tube 53g are fixedly attached to the corresponding members by adhering, welding, or the like to form a four-layer structure as a unit. This makes the inserting barrel body 53b a flexible tube body having a predetermined rigidity.

The inserting barrel body 53b may be formed as a barrel body formed of a single member as long as a predetermined rigidity and a predetermined flexibility can be sufficiently obtained. Furthermore, the inserting barrel body 53b may have a coating such as Teflon® process for example, to enhance slidability of the outer and inner circumferential surfaces thereof. The inserting barrel body 53b is provided with the above described deviation preventing section 54 which is a hollow disk including a synthetic resin such as silicon, at the proximal end part.

The deviation preventing section 54 has a hole having a diameter which is smaller than that of the inserting barrel body 53b. The elastic deformation of the deviation preventing section 54 yields a predetermined holding strength which causes the deviation preventing section 54 to be closely fixed to the inserting barrel body 53b. Depending on the position of the deviation preventing section 54, the length of the insertion tube 53 between the distal end and the proximal end to be inserted into a body cavity can be desirably set.

The inserting barrel body 53b has a three-layer structure at the proximal end part as shown in FIG. 5, which is formed of the blade 53e, the flex tube 53f, and the inner tube 53g, and is fixedly attached to the circular ring shaped cap 60 by adhering or the like. The retaining tube 55 is screwed with the cap 60 at the proximal end of the inserting barrel body 53b of the insertion tube 53.

The tube main body 58 has an opening 58a formed in the distal end thereof, which is a distal end opening of the distal end insertion tube 53a. The opening 58a forms an opening of the insertion assisting tool 11 from which the inserting section 6A is protruded.

The tube main body 58 has the retaining tube 55 at the proximal end thereof, and the retaining tube 55 has the female screw 55a to which the male screw 56a of the first securing ring 56 is screwed to fix the first securing ring 56 thereto.

The clamp ring 59 through which the guide tube toward the distal end 13 is inserted is fitted into the first securing ring 56, and also the second securing ring 57 is fixed to the first securing ring 56. In other words, the screwed female screw 56b of the first securing ring 56 and the male screw 57a of the second securing ring 57 make the second securing ring 57 fixed to the first securing ring 56.

At this point, the clamp ring 59 is fitted and fixed between the end surface of the first securing ring 56 and the end surface of the second securing ring which are opposed to each other. The clamp ring 59 has inward flanges 59a and 59b which extend inward about an axis at the center of the inner circumferential surface and at the proximal end of the clamp ring 59.

The inward flanges 59a and 59b, as shown in FIG. 6 and FIG. 7, lock the corrugated convex and concave parts of the guide tube toward the distal end 13. This allows the guide tube toward the distal end 13 to be connected to the insertion assisting tool 11. The guide tube toward the distal end 13 is compressed against the surface of the proximal end of the stepped part of the first securing ring 56 at the distal end part F of FIG. 6, so that the guide tube toward the distal end 13 is connected to the insertion assisting tool 11 in a watertight way. That is, the insertion assisting tool 11 and the guide tube toward the distal end 13 form a tube path which has the opening 58a of the tube main body 58 as a distal end opening.

FIG. 8 shows the insertion assisting tool 11 into which an inserting section 6A is inserted. As shown in FIG. 8, the insertion assisting tool 11 is configured to allow the inserting section 6A to be retracted to the position CO (the character CO which is shown in FIG. 8) where an image of the distal end section 100a and around the distal end section 100a of the projecting part 100 can be picked up, when the inserting section 6A is retracted.

In other words, the position CO is the position where an image of a region such as the rectum can be observed by the image pickup unit 27 at the distal end section 8 of the inserting section 6A (which is also called as an observation end position). Also, the position CO is the removal end position where the inserting section 6A is removed from a body cavity such as the large intestine.

The removal end position CO is set depending on the viewing angle θ of the image pickup unit 27 which is provided at the distal end section 8 of the inserting section 6A. At the same time, the length of the projecting part 100 which projects in the insertion direction is set depending on the above described removal end position CO and the viewing angle θ of the image pickup unit 27.

In this embodiment, while an endoscope image is displayed on the monitor 4 based on an image pickup signal captured by the image pickup unit 27, when the projecting part 100 (including the distal end part 100a) which is a part of the distal end part of the insertion assisting tool 11 is displayed in the endoscope image, an operator determines that the distal end section 8 of the inserting section 6A has reached the removal end position CO, and stops the retraction of the inserting section 6A. So, when the inserting section 6A is retracted, a region near the distal end part of the insertion assisting tool 11 such as rectum can be observed, and at the same time, the removal of the inserting section 6A from the large intestine can be recognized in a quick and reliable way.

Next, with reference to FIG. 9 to FIG. 13, the housing case main body 12 will be explained below.

As shown in FIG. 9, the housing case main body 12 includes: a main body 12a having first and second plate members 61 and 62 which are two plate-shaped parts, with the surfaces thereof being arranged in parallel to form a space 12b therebetween into which the inserting section 6A is housed; a first opening 68A provided to the main body 12a for protruding the inserting section 6A from the space 12b or introducing the inserting section 6A into the space 12b, and a second opening 68B provided to the main body 12a for protruding the distal end part of the inserting section 6A from the space 12b; and two guide tube fixing members 64 and 65.

The housing case which constitutes an endoscope inserting section with a case of the present invention includes the housing case main body 12, the guide tubes 13 and 14, and the insertion assisting tool 11.

The main body 12a is configured with the two, first and second, plate members 61 and 62 such as acrylic boards which are rectangular and transparent or semitransparent to be optically transparent, and a plurality of frame members 63a to 63f which are fixedly attached to surfaces of each of the two first and second plate members 61 and 62 to separate the two plate members 61 and 62 by a predetermined distance in an opposite relationship to each other to form the space 12b therebetween.

The above described plate members 61 and 62 may be semitransparent or colored plated which are optically transparent so that it is not easy to find contaminants adhered to the rotatable barrel body 51 and a rotation of the rotatable barrel body 51 can be visually recognized.

The distance between the opposing surfaces of each of the first plate member 61 and the second plate member 62 is smaller than twice the size of the outer diameter at the convex part position where the helical-surface shaped part 51a of the rotatable barrel body 51 is formed.

Each of the two first and second plate members 61 and 62 has L-shaped legs 66 at the corners on a surface which is on the opposite side of the opposing surface. Each of the legs 66 is fixedly attached to the surface of each of the first and second plate members 61 and 62 by adhering or the like, with the corner of the leg 66 being aligned with one of the four corners of each of the plate members 61 and 62.

The frame members 63a to 63f having an equal size in the thickness direction are fixedly attached to the periphery parts of the surfaces which are on the opposite side of the surfaces having the legs attached thereto of first and second plate members 61 and 62, by adhering or the like. That is, the first and second plate members 61 and 62, as described above, are separated by the thickness of each of the frame members 63a to 63f, and are fixed so that the plate surfaces are in parallel to each other.

The frame members 63a to 63f are generally rectangular prisms which individually have a predetermined length, and as shown in FIG. 10, are provided on the four peripheral sides of each of the first and second plate members 61 and 62 to surround the first and second plate members 61 and 62. That is, the housing case main body 12 includes the space 12b which is formed by the two first and second plate members 61 and 62 and each of the frame members 63a to 63f.

Also, between an end of the frame member 63a and an end of the frame member 63b, and between an end of the frame member 63c and an end of the frame member 63d, there are formed first and second openings 68A and 68B respectively so that the above described inserting section 6A can be inserted therethrough.

The frame members 63a to 63d for forming the first and second openings 68A and 68B are individually provided with an inner corner having a smooth curved surface 69 to make the inserting section 6A smoothly pass therethrough.

The first and second openings 68A and 68B are individually provided at side surfaces of the frame members 63a to 63d so that the end of each of the openings 68A and 68B is disposed at a position separated by a predetermined distance from one of the corners of the main body 12a. That is, the first and second openings 68A and 68B are configured so that the inserted inserting section 6A contacts the inner circumferential surface of the main body 12a to the minimum extent possible, and also the inserted inserting section 6A passes therethrough while smoothly rotating. Any damage to the inserted inserting section 6A is also prevented.

The housing case main body 12 is provided with the first opening 68A and the second opening 68B in a way that a central axis of a hole of the first opening 68A and a central axis of a hole of the second opening 68B are not coaxially disposed in an imaginary plane for the space 12b which is seen from the top.

Specifically, as shown in FIG. 9, the first opening 68A and the second opening 68B are arranged so that each of the central axes of the holes is positioned orthogonally relative to each other.

That is, this configuration allows the inserting section 6A which is inserted through the first opening 68A or the second opening 68B to be bent to be housed in the space 12b of the housing case main body 12.

Meanwhile, each of the guide tube fixing members 64 and 65 is fixed to one side surface of the housing case main body 12 by using fixing members 67 including a bolt and a nut to connect between the frame members 63a and 63b or the frame members 63c and 63d at the positions for the above described first and second openings 68A and 68B.

Specifically, the guide tube fixing members 64 and 65 are, as shown in FIG. 11, formed of metals (may be formed of a rigid materials including a synthetic resin or plastic), and have barrel bodies 64a and 65a and rectangular plate members 64b and 65b which are mounted to one end of the barrel bodies 64a and 65a, respectively.

The guide tube fixing members 64 and 65 are, as shown in FIG. 11 and FIG. 13, fixed by using the fixing members 67 which are inserted through threaded holes 67a formed in each of the frame members 63a to 63d (only the threaded holes 67a of the frame members 63a and 63b are shown in FIG. 12).

Each of the guide tube fixing members 64 and 65 are fixed so that the holes of the barrel bodies 64a and 65a are aligned with the center of the first and second openings 68A and 68B respectively to be in communication with the space 12b of the housing case main body 12.

Therefore, each of the guide tube fixing members 64 and 65 are arranged depending on the positions of the first and second openings 68A and 68B.

That is, each of the guide tube fixing members 64 and 65 are, as shown in FIG. 9, arranged with the guide tube fixing member (hereinafter, also referred to as a first guide tube fixing member) 64 being fixed around the bottom left corner at the side surface at the side surface facing toward the lower side the housing case main body 12 in FIG. 9, and the guide tube fixing member (hereinafter, also referred to as a second guide tube fixing member) 65 being fixed around the bottom right corner at the side surface facing toward the left side of the housing case main body 12 in FIG. 9. Each of the guide tube fixing members 64 and 65 will not be arranged around the same corner.

Also, between the plate members 64b and 65b of the guide tube fixing members 64 and 65 and the frame members 63a to 63d, there are individually interposed rubber plates 76 to keep them water tight (see FIG. 15, FIG. 16, and FIG. 18).

The first guide tube fixing member 64 is connected to one end of the guide tube toward the distal end 13, the other end of the guide tube 13 being connected to the above described insertion assisting tool 11. While, the second guide tube fixing member 65 is connected to one end of the guide tube toward the operating section 14, the other end of the guide tube 14 being connected to the motor box 16 of the operating section 7.

FIG. 14 shows a connection structure between the first guide tube fixing member 64 and the guide tube toward the distal end 13.

Specifically, as shown in FIG. 14, the barrel body 64a of the first guide tube fixing member 64, which is a coupling section to couple the housing case main body 12 to the guide tube toward the distal end 13, is provided a female screw part 64c at an inner circumferential surface of the proximal end thereof.

In the barrel body 64a, in order, a first impelling force generating member 70a, a first holding ring 71, a second impelling force generating member 70b, and a second holding ring 72 are arranged in an array. The third securing ring 73 is screwed on the barrel body 64a, with the female screw part 64c of the barrel body 64a being threadedly engaged with a male screw part 73a on the outer circumferential surface at the midst of the third securing ring 73.

The third securing ring 73 is a generally cylindrical metal ring (may be a rigid cylindrical body, including a synthetic resin and plastic, for example) which has an outer circumferential part projected in the direction of the outer diameter at the proximal end thereof. The third securing ring 73 has a female screw part 73b in the inner circumferential surface of the proximal end thereof.

A clamp ring 74, through which the guide tube toward the distal end 13 is inserted, is fitted in the third securing ring 73, and also a fourth securing ring 75 is fixed to the third securing ring 73. In other words, when the female screw part 73b of the third securing ring 73 is screwed to the male screw part 75a of the fourth securing ring 75, the fourth securing ring 75 is fixed to the third securing ring 73.

At this point, the clamp ring 74 is fitted and fixed between the end surface of the third securing ring 73 and the end surface of the fourth securing ring 75 which are opposed to each other. The clamp ring 74 has inward flanges 74a and 74b which extend inward about an axis at the center of the inner circumferential surface and at the proximal end of the clamp ring 74.

The inward flanges 74a and 74b, as shown in FIG. 15 and FIG. 16, lock the corrugated convex and concave parts of the guide tube toward the distal end 13. This allows the guide tube toward the distal end 13 to be connected to the guide tube fixing member 64 via the third securing ring 73 and the fourth securing ring 75.

The guide tube toward the distal end 13 is compressed against the surface of the distal end of the stepped part in the third securing ring 73 at the proximal end part G of FIG. 16, so that the guide tube toward the distal end 13 is connected to the guide tube fixing member 64 and the third securing ring 73 in a watertight way.

The first guide tube fixing member 64 has two impelling force generating members 70a and 70b disposed therein which are impelling force generating sections formed of plate members of a generally disk-like shape with a predetermined thickness.

Each of the impelling force generating members 70a and 70b has, as shown in FIG. 17, a hole 77 formed in the generally center part thereof. The hole 77 is of a generally rectangular shape having round corners. The round shape of the corners is provided to prevent the impelling force generating members 70a and 70b from breaking.

The impelling force generating members 70a and 70b are formed of a material which is more flexible than that of the insertion tube 53 of the above described tube main body 58, such as synthetic natural rubber and silicon rubber. For example, the material is an elastic member having a hardness in a range of A20 to A90 which is examined by a spring type durometer hardness test Type A (JIS-K6253, International Organization for Standardization; ISO7619).

Now, with reference to FIG. 15, FIG. 16, and FIG. 18, each arrangement of the two impelling force generating members 70a and 70b, the two holding rings 71 and 72, and the third securing ring 73 in the first guide tube fixing member 64 will be explained below.

First, the first impelling force generating member 70a is disposed in contact with an end surface of the plate member 64b at the connection between the barrel body 64a and the plate member 64b of the first guide tube fixing member 64. Then, the holding ring 71, the second impelling force generating member 70b, and the holding ring 72 are housed in this order in the barrel body 64a of the first guide tube fixing member 64. That is, in this state, in the barrel body 64a, the first impelling force generating member 70a, the holding ring 71, the second impelling force generating member 70b, and the holding ring 72 are disposed in this order from the proximal end.

Then, the third securing ring 73 is inserted into the proximal end opening of the barrel body 64a to make the male screw part 73a of the third fixing ring 73 threadedly engaged with the female screw part 64c of the barrel body 64a. In this state, the first impelling force generating member 70a is sandwiched between the plate member 64b and the holding ring 71 with the peripheral part of the first impelling force generating member 70a being in contact with the end surface of the plate member 64b and the proximal end peripheral part of the holding ring 71.

Also, the second impelling force generating member 70b is sandwiched between the holding ring 71 and the holding ring 72 with the peripheral part of the second impelling force generating member 70b being in contact with the distal end peripheral part of the holding ring 71 and the proximal end peripheral part of the holding ring 72. In this configuration, the holding ring 72 is pressed against the proximal end peripheral part of the third securing ring 73 at the distal end peripheral part thereof.

The barrel body 64a, the two holding rings 71 and 72, and the third securing ring 73 are individually set to have a length in each axial direction thereof to sandwich the first and second impelling force generating members 70a and 70b at a predetermined pressure. And the distance between the opposing surfaces of the first impelling force generating member 70a and the second impelling force generating member 70b is equal to the length in the axial direction of the holding ring 71. So, the first impelling force generating member 70a and the second impelling force generating member 70b are set to be separated by a predetermined distance depending on the length in the axial direction of the holding ring 71.

The distance between the opposing surfaces of the first impelling force generating member 70a and the second impelling force generating member 70b is set to be generally equal to the pitch of the convex and concave parts formed on the helical-surface shaped part 51a of the rotatable barrel body 51. In other words, the length of in the axial direction the holding ring 72 and the plate thickness of each of the impelling force generating members 70a and 70b is set in response to one pitch of the convex and concave parts formed on the helical-surface shaped part 51a.

Each of the end surfaces of the peripheral parts pressing the impelling force generating members 70a and 70b of the two holding rings 71 and 72 may be roughly textured as a misalignment preventing section such as an uneven surface for preventing a misalignment, in order to maintain a reliable sandwich configuration in contacting the first and second impelling force generating members 70a and 70b. In other words, since there will be generated a large frictional force between the rough end surfaced of the two holding rings 71 and 72 and the surfaces of the first and second impelling force generating members 70a and 70b which are pressed against the rough surfaces, the first and second impelling force generating members 70a and 70b are reliably sandwiched in the corresponding guide tube fixing member 64 without any misalignment caused by an exterior force.

Through the holes 77 of the first and second impelling force generating members 70a and 70b which are arranged in the guide tube fixing member 64 as described above, as shown in FIG. 18, the inserting section 6A housed in the housing case main body 12 is inserted. At this point, the first and second impelling force generating members 70a and 70b press the rotatable barrel body 51 at a part of the inner circumferential surface of each of the holes 77 by using its elastic force.

Each of the holes 77 of the first and second impelling force generating members 70a and 70b has a longitudinal length L1 and a lateral length L2 in the relationship L1>L2, of course. The rotatable barrel body 51 which forms the outer contour of the inserting section 6A inserted through the holes 77 has a diameter at the concave part L3 and a diameter at the convex part L4 in the relationship L3<L4, of course.

In this embodiment, the holes 77 and the convex and concave parts included in the helical-surface shaped part 51a of the rotatable barrel body 51 individually have sizes which are designed so that the lateral length L2 of the holes 77 is a little shorter than the diameter length L3 of the concave part of the helical-surface shaped part 51a (L2<L3), and the longitudinal length L1 of the holes 77 is longer than the diameter length L4 of the convex part of the helical-surface shaped part 51a (L1>L4).

In other words, the first and second impelling force generating members 70a and 70b press the concave part of the helical-surface shaped part 51a generally at the two lateral surfaces of each hole 77 where the rotatable barrel body 51 contacts, by utilizing the elastic deformation. When the rotatable barrel body 51 rotates about the axis, frictional force is generated between the rotatable barrel body 51 and the first and second impelling force generating members 70a and 70b, and at the same time the rotatable barrel body 51a is advanced or retracted at each hole 77 of the first and second impelling force generating members 70a and 70b by the screw action.

Alternatively, the above described lateral length L2 of the holes 77, the diameter L3 of the concave part of the helical-surface shaped part 51a, and the diameter L4 of the convex part of the helical-surface shaped part 51a may be designed in the relationship L3<L2<L4 so that the rotating rotatable barrel body 51 can be advanced or retracted by the screw action.

When the inserting section 6A is subjected to a resistance above a predetermined amount in a region to be examined such as the intestinal wall of a body cavity, the rotatable barrel body 51 idly rotates at the position relative to the first and second impelling force generating members 70a and 70b not to be advanced in the deeper part of the site.

That is, as described above, since each hole 77 of the first and second impelling force generating members 70a and 70b has the longitudinal length L1 which is set to be longer than the diameter length L4 of the convex part of the helical-surface shaped part 51a (L1>L4) and the rotatable barrel body 51 is inserted in the direction of the longitudinal side of the hole 77 with a clearance therebetween, an application of a predetermined force (frictional force in the advancing direction) on the rotatable barrel body 51 makes the rotatable barrel body 51 rotate idly at the position of the hole 77, which prohibits the rotatable barrel body 51 from advancing into the deeper part of the subject site.

This prevents an excess pushing of the inserting section 6A by the generated impelling force so that unnecessary load is not applied to a region to be examined such as a body cavity by the insertion of the inserting section 6A.

As means for generating an impelling force for the rotatable barrel body 51, in stead of the above described plate-shaped impelling force generating members 70a and 70b, one impelling force generating member 70c which includes an elastic member such as a generally barrel-shaped silicon rubber may be used as shown in FIG. 19 and FIG. 20.

Specifically, the impelling force generating member 70c has a hole 77a of a generally square shape having round corners. In this case also, the round shape of the corners is provided to prevent the impelling force generating member 70c from breaking. The impelling force generating member 70c has a length in the axial direction which is set to be longer than one pitch of the convex and concave parts formed on the helical-surface shaped part 51a of the rotatable barrel body 51, for example about 6 mm.

The hole 77a of the impelling force generating member 70c also has a minimum diameter which is set to be a little smaller than the outer diameter of the convex part formed on the helical-surface shaped part 51a of the rotatable barrel body 51.

Contrary to this embodiment, the hole 77a of the impelling force generating member 70c may have a minimum diameter which is set to be a little larger than the outer diameter of the convex part formed on the helical-surface shaped part 51a of the rotatable barrel body 51, so that a contact of the rotatable barrel body 51 with either the upper surface or the lower surface of the hole 77a can apply an impelling force to the rotating rotatable barrel body 51.

The impelling force generating member 70c is provided in the barrel body 64a of the first guide tube fixing member 64. That is, as described above, the impelling force generating member 70c is arranged in the barrel body 64a with a proximal end surface at the peripheral part of one end being in contact with an end surface of the plate member 64b of first guide tube fixing member 64 which is adjacent to the inside of the barrel body 64a, while the distal end surface at the peripheral part of the other end being pressed against the proximal end peripheral part of the third securing ring 73.

And the impelling force generating member 70c presses the helical-surface shaped part 51a of the rotatable barrel body 51 which is inserted through the hole 77a by means of the elastic force. That is, the rotating rotatable barrel body 51 is advanced or retracted by the screw action which is caused by the friction between the convex part of the helical-surface shaped part 51a and the inner surface of the hole 77a in the impelling force generating member 70c.

The second guide tube fixing member 65 is not provided with the impelling force generating members 70a and 70b therein, and is connected to the guide tube toward the operating section 14 generally in the same structure as the first guide tube fixing member 64.

Specifically, as shown in FIG. 21, the second guide tube fixing member 65 is directly connected to the above described fourth securing ring 75. In other words, the second guide tube fixing member 65 is connected to the guide tube toward the operating section 14 by holding the clamp ring 74, which is engaging the guide tube toward the operating section 14, together with the fourth securing ring 75, as in the case of the above described third securing ring 73.

The barrel body 65a of the second guide tube fixing member 65 includes a proximal end having an inner circumferential surface in which the female screw part 65c is formed to be threadedly engaged with the male screw part 75a of the fourth securing ring 75.

In this configuration also, one end of the guide tube toward the operating section 14 is compressed against the end surface of the second guide tube fixing member 65 in contact with the clamp ring 74, so that the guide tube toward the operating section 14 is connected to the second guide tube fixing member 65 in a watertight way. The other end of the guide tube toward the operating section 14 which is connected to one end of the second guide tube fixing member 65 is connected to the connector cover 15.

Next, with reference to FIG. 22 to FIG. 25, a connection between the guide tube toward the operating section 14 and the connector cover 15 will be explained below.

As shown in FIG. 21 and FIG. 22, the guide tube toward the operating section 14 is fitted into a fifth securing ring 78 which includes a generally cylindrical metal ring (may be a rigid cylindrical body including a synthetic resin, plastic, or the like), and a clamp ring 81 which is threadedly engaged with a connecting barrel body 79 including a synthetic resin to lock the outer circumferential of the proximal end part thereof.

The clamp ring 81 has the same configuration as those of the clamp rings 59 and 74 which lock the both ends of the above described guide tube toward the distal end 13 and the distal end part of the guide tube toward the operating section 14, respectively, and so will not be described in detail below.

The fifth securing ring 78 has a projected outer circumferential part in the direction of the outer diameter at the midst part thereof, and includes a proximal end having an outer circumferential surface on which a male screw part 78a is formed. The connecting barrel body 79 has a projected outer circumferential part in the direction of the outer diameter at the distal end part where a female screw part 79a is formed in the inner circumferential surface thereof, extends toward the proximal end in circles around at generally equal distances, and has a plurality of locking sections 80 which allow the connecting barrel body 79 to be removably attached to the connector cover 15.

In other words, the fifth securing ring 78 and the connecting barrel body 79 are connected to each other by the engagement between the male screw part 78a and the female screw part 79a, and the clamp ring 81 is fitted and held in the connection. In this state, the proximal end part of the guide tube toward the operating section 14 is compressed, and the outer circumferential surface at the proximal end is in contact with and pressed by the end surface of the connecting barrel body 79. This configuration allows the guide tube toward the operating section 14 to be connected with the fifth securing ring 78 and the connecting barrel body 79 in a watertight way.

The connecting barrel body 79 connected to the connector cover 15 has the locking section 80 which is connected to the connector cover 15. Specifically, the connector cover 15 is provided with a connecting section 82 which includes a cylindrical body having outward flanges 82a at the distal and proximal end parts thereof and notches 82b in the barrel body along the axial direction (see FIG. 23).

Onto the connecting section 82, the plurality of locking sections 80 of the connecting barrel body 79 are fitted to be connected thereto. The plurality of locking sections 80 have projections 80a which extend inward of the connecting barrel body 79 at each proximal end. So, the locking between the projections 80a and the outward flanges 82a at the proximal end part of the connecting section 82 allows the connecting barrel body 79 and the connector cover 15 to be removably connected.

Since each projection 80a of the locking sections 80 simply engages the outward flanges 82a of the connecting barrel body 79, the connecting barrel body 79 is rotatable about the axis relative to the connector cover 15. Therefore, the guide tube toward the operating section 14 coupled to the connecting barrel body 79 is also rotatably connected relative to the connector cover 15.

In addition, as shown in FIG. 23, the proximal end part of the rotatable barrel body 51 is fixedly attached to the cap 83 by using an adhesive or the like. The cap 83 is connected to the distal end part of a rotary shaft 84 by using a screw. The rotary shaft 84 (not shown) is rotatably supported in the connector cover 15.

Upon a connection between the connector cover 15 and the motor box 16 (see FIG. 1), a gear (not shown) in the rotary shaft 84 and a gear (not shown) in motor box 16 are meshed to each other. Then, the driving power of the motor is transmitted to each of the gears which, via the rotary shaft 84 and the cap 83, causes the rotatable barrel body 51 to rotate about the axis.

Next, with reference to FIG. 25, each of the guide tubes 13 and 14 will be explained below.

The guide tubes 13 and 14 are, as described above, accordion tubes with uneven outer and inner circumferential surfaces, so-called corrugated tube bodies, including a synthetic resin which is transparent or semitransparent to be optically transparent. The guide tubes 13 and 14 are flexible, and have an inner diameter size which generally does not change and are not buckled even when the guide tubes 13 and 14 are bent, due to the uneven contour.

The guide tubes 13 and 14 have a minimum inner diameter L6 at the position of a convex part which projects in the direction of the inner diameter. The minimum inner diameter L6 of the guide tube is set to be larger than the length L5 which is the outer size at the position of the convex part included in the helical-surface shaped part 51a of the rotatable barrel body 51 (L6>L5). In other words, the guide tubes 13 and 14 are tube bodies through which the rotatable barrel body 51 can be inserted with room.

When a power for rotating the rotatable barrel body 51 about the axis is applied, a sliding of the material and shear stress are caused, and torsional stress is generated inside of the rotatable barrel body 51. Depending on the torsional stress, the flexible rotatable barrel body 51 may be curled into a ring shape.

To avoid the curling, the minimum inner diameter L6 of the guide tubes 13 and 14 is set to be less than twice of the length L5, which is the outer diameter size at the position of the convex part included in the helical-surface shaped part 51a of the rotatable barrel body 51 (L4<2L5). The guide tubes 13 and 14 have a predetermined hardness which effectively resists any deformed movement (swing) of the rotatable barrel body 51 which is caused by the torsional stress.

This is also true for the case of the above described housing case main body 12, and as shown in FIG. 26, the distance L7 between the first and second plate members 61 and 62 is set to be less than twice of the length L5, which is the outer diameter size at the position of the convex part of the rotatable barrel body 51, (L7<2L5) to prevent the rotatable barrel body 51 from curling into a ring shape caused by the torsional stress.

That is, each of the frame members 63a to 63f is set to have a length L7 in the height direction so that the two first and second plate members 61 and 62 are separated by the distance L7 to be parallel to each other. Of course, the distance L7 between the first and second plate members 61 and 62 is longer than the length L5 which is the outer size at the position of the convex part included in the helical-surface shaped part 51a of the rotatable barrel body 51 (L7>L5).

The above described endoscope system 1 of this embodiment is configured to include the elements from the distal end section 8 to the connector cover 15 as the inserting section with a case 6 (see FIG. 1), and the inserting section with a case 6 is a disposable type which is discarded after use.

The inserting section with a case 6 of this embodiment is disposable, but a used inserting section with a case 6 may be reused after a sufficient disinfection and sterilization.

In this embodiment, the first and second impelling force generating members 70a and 70b are provided in the first guide tube fixing member 64 of the housing case main body 12, but as illustrated as a modification in FIG. 27, the first and second impelling force generating members 70a and 70b may be provided in the insertion assisting tool 11. Since the latter structure is especially effective in this embodiment, the structure of the modified example will be explained below with reference to FIG. 27.

As shown in FIG. 27, the insertion assisting tool 11 is provided with the first and second impelling force generating members 70a and 70b having the above described structure at the inside near the deviation preventing section 54. The first and second impelling force generating members 70a and 70b are generally mounted in the same way as that in the first guide tube fixing member 64.

The holding rings 71 and 72 which press the first and second impelling force generating members 70a and 70b are held between the first and second impelling force generating members 70a and 70b and the third securing ring 73.

In the insertion assisting tool 11 which is provided with the first and second impelling force generating members 70a and 70b in this configuration, if the inserting section 6A is retracted by using a conventional method for removing an inserting section, a part of the inserting section 6A which does not have the rotatable barrel body 51 is brought in contact with the first and second impelling force generating members 70a and 70b. In this state, if the inserting section 6A is again moved to be inserted by rotating the rotatable barrel body 51 into a body cavity, the first and second impelling force generating members 70a and 70b may idly rotate at the position and not able to cause the rotatable barrel body 51 to be inserted into the body cavity again.

However, the insertion assisting tool 11 of this embodiment is configured so that a part of the inserting section 6A which does not have the rotatable barrel body 51 is not brought in contact with the first and second impelling force generating members 70a and 70b.

In other words, as shown in FIG. 27, when the rotatable barrel body 51 is in contact with the first and second impelling force generating members 70a and 70b, the distal end section 8 of the inserting section 6A is positioned at the above described removal end position CO. That is, the inserting section 6A is caused to stop at the removal end position CO not to be retracted beyond the removal end position CO by using a removing method of this embodiment which will be explained later.

So, the inserting section 6A can be inserted into a body cavity again without the above described idle rotation of the first and second impelling force generating members 70a and 70b.

Next, a method for removing an inserting section according to the present invention will be explained below with reference to FIG. 28 to FIG. 32. In the following explanation, an examination of large intestine is used as an example. Also in the following, the first and second impelling force generating members 70a and 70b is provided in the first guide tube fixing member 64. Of course, the first and second impelling force generating members 70a and 70b may be provided in the insertion assisting tool 11.

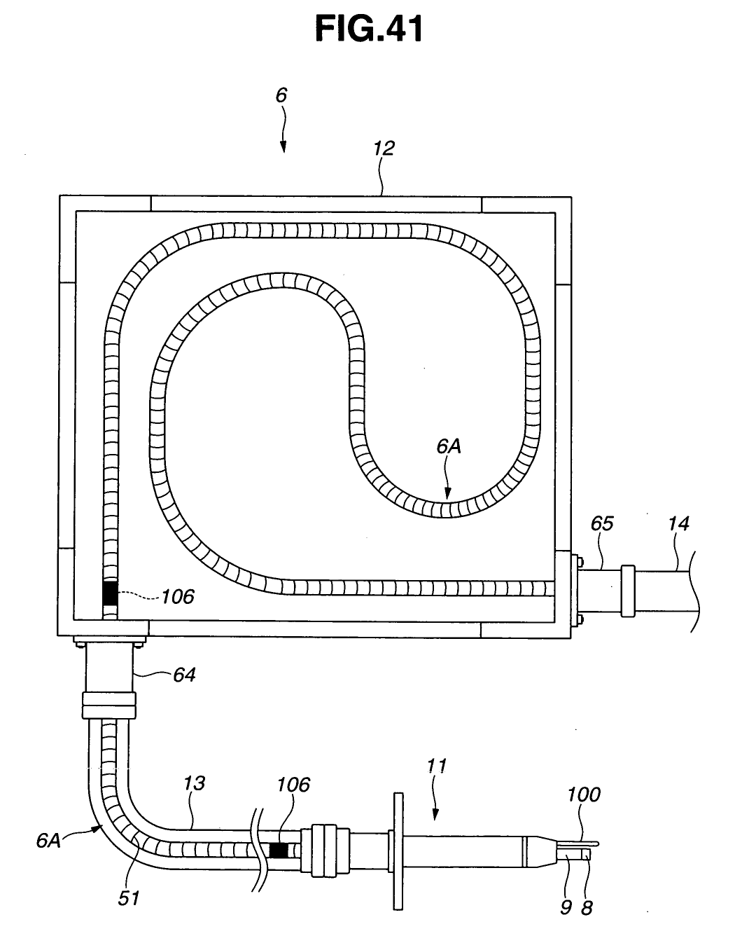

First, in inserting the inserting section 6A of the endoscope 2 into a part of the large intestine such as cecum, a healthcare practitioner prepares the endoscope system 1 of this embodiment shown in FIG. 1 in a predetermined manner. First, an operator, who is a doctor in this embodiment, inserts the insertion assisting tool 11 into a patient who is lying on a bed, via anus. The inserting section 6A is housed in a form of loop in the housing case main body 12 as shown in FIG. 41.

As for the insertion assisting tool 11, as shown in FIG. 28, the deviation preventing section 54 is brought in contact with the buttocks 510 near the anus 501 of the patient so that only the insertion tube 53 is inserted into the rectum 502 via anus 501. That is, the deviation preventing section 54 prevents the entire insertion assisting tool 11 from being inserted into the rectum 502. At this point, the operator desirably attaches the deviation preventing section 54 to the buttocks 510 by using a tape or the like.

In this state, when the endoscope system 1 is set up, the operator holds the holding section 17 of the operating section 7, and rotates the rotatable barrel body 51 of the inserting section 6A in a predetermined direction about the axis by operating the foot switch 25 of FIG. 1 by foot or by operating the advancement/retraction switch at the main operating section 18 by hand.

The two impelling force generating members 70a and 70b provided in the guide tube fixing member 64 of the housing case main body 12 are, as shown in FIG. 18, constantly pressed against the concave part at the helical-surface shaped part 51a of the rotatable barrel body 51. In other words, the rotatable barrel body 51 is under a predetermined frictional resistance at the generally center of each hole 77 of the impelling force generating members 70a and 70b.

In the contacting state, the operator causes the motor box 16 provided in the operating section 7 to be driven for rotation by the above described operation by foot or by hand. Then, a torque is transmitted from the proximal end part to the distal end part of the rotatable barrel body 51, which causes the entire rotatable barrel body 51 to rotate in a predetermined direction about the axis as shown by an arrow of FIG. 28.

The rotation produces an impelling force for advancing the rotatable barrel body 51 as the male screw moves relative to the female screw at the connection between each hole 77 of the impelling force generating members 70a and 70b and the concave part at the helical-surface shaped part 51a of the rotating rotatable barrel body 51.

In the rotating rotatable barrel body 51 with the impelling force, the cap 50 fixedly attached to the distal end of the rotating rotatable barrel body 51 presses the third cap 48 at the proximal end of the bending section 9. This allows the distal end section 8 and the inserting section 6A including the bending section 9 to be advanced into a deeper part of the large intestine via the guide tube toward the distal end 13 and the insertion assisting tool 11 by the impelling force of the rotatable barrel body 51.

At this point, the operator can make the inserting section 6A advanced into a deeper part of the large intestine only under the impelling force from each of the impelling force generating members 70a and 70b while the operator gently holds the retaining tube 55 of the insertion assisting tool 11 without holding and pushing the inserting section 6A.

The inserting section 6A can be held in the longitudinal direction in a stable condition and is effectively applied with the impelling force for advancing, since the two impelling force generating members 70a and 70b are provided in the guide tube fixing member 64 with a predetermined distance therebetween.

In addition, the helical-surface shaped part 51a formed on the outer surface of the rotatable barrel body 51 is brought in contact with the intestinal wall. At this point, the helical-surface shaped part 51a formed on the rotatable barrel body 51 and the folds of the intestinal wall are connected to each other in the same relationship as that of a male screw and a female screw. So, the rotatable barrel body 51 is smoothly advanced under the impelling force caused by each of the impelling force generating members 70a and 70b in the guide tube fixing member 64 and the impelling force caused by the contact with the folds of the intestinal wall.

Then, the inserting section 6A under the impelling forces is advanced toward the sigmoid colon 503 from the rectum 502. And as shown in FIG. 29, the distal end section 8 and the bending section 9 reach the sigmoid colon 503. At this point, the operator operates the two bending knobs 19 at the main operating section 18 (see FIG. 1) while watching an endoscope image displayed on the monitor 4, to bend the bending section 9 by following the curving sigmoid colon 503.

The bending of the bending section 9 enables the operator to advance the inserting section 6A under the impelling forces into the sigmoid colon 503, where it is said to be difficult to insert the section, to smoothly pass the distal end section 8 and the bending section 9 through the sigmoid colon 503.

As the inserting section 6A is inserted into a deeper part of the large intestine, the inserting section 6A is constantly under the impelling force caused by the impelling force generating members 70a and 70b in the guide tube fixing member 64, and the length of the helical-surface shaped part 51a which is in contact with the intestinal wall gets longer.

Therefore, a stable impelling force toward a deeper part of the large intestine can be obtained even when a part of helical-surface shaped part 51a is in contact with the folds of the sigmoid colon 503 or the inserting section 6A is bent in a complicated way. Moreover, since the inserting section 6A is sufficiently flexible, the inserting section 6A can be smoothly advanced along the intestinal wall without changing the running state of the sigmoid colon 503 which readily changes its position.

After passing sigmoid colon 503, the inserting section 6A which includes the rotating rotatable barrel body 51 is smoothly advanced along a flexion between the sigmoid colon 503 and the less movable descending colon 504, the splenic flexure 505 between the descending colon 504 and the more movable transverse colon 506, and the wall of the hepatic flexure 507 between the transverse colon 506 and the ascending colon 508, and as shown in FIG. 30, reaches a part near the cecum 509 which is the target region without changing the running state of the large intestine.

In this inserting operation, when the distal end section 8 reaches each flexion (the splenic flexure 505 and the hepatic flexure 507), the operator operates the two bending knobs 19 at the main operating section 18 while watching an endoscope image displayed on the monitor 4, to bend the bending section 9 by following the curving state of each site, in the same way as described above.

The operator once stops the rotation of the rotatable barrel body 51 by the above described operation by foot or by hand when the operator determines that the distal end section 8 has reached a part near the cecum 509 by using the endoscope image displayed on the monitor 4. Then the operator operates to cause the rotatable barrel body 51 to rotate in a direction which is opposite to that in which the rotatable barrel body 51 was rotated in about the axis for insertion, by the foot operation using the foot switch 25 or the hand operation using the advancement/retraction switch at the main operating section 18.

In other words, the rotatable barrel body 51 is rotated in the opposite direction to that for insertion in order to check the large intestine, while the inserting section 6A being caused to retract in the direction for removing it from the deep part of the large intestine and a part near the cecum 509. At this point also, the operator can make the inserting section 6A retracted under the impelling forces caused by the connection between the rotatable barrel body 51 and the impelling force generating members 70a and 70b in the guide tube fixing member 64 and the folds of the intestinal wall without touching inserting section 6A.

Because the uneven part 50a of the cap 50 at the distal end of the rotatable barrel body 51 is engaged with the projection 48a of the third cap 48 at the proximal end of the bending section 9, the distal end section 8 and the bending section 9 are pulled by the rotatable barrel body 51, and thereby the entire inserting section 6A is retracted under the impelling force of the rotatable barrel body 51.

In the method for removing an inserting section of this embodiment, an operator causes the inserting section 6A to be retracted while watching an endoscope image displayed on the monitor 4.

And the operator stops the retraction of the inserting section 6A by determining that the distal end section 8 of the inserting section 6A is already removed from the large intestine, when the endoscope image on the monitor 4 displays the distal end part of the insertion assisting tool 11. An example of the screen display on the monitor 4 is shown in FIG. 32.

That is, as shown in FIG. 32, when the endoscope image 4A displayed on the monitor 4 includes the projecting part 100 of the insertion assisting tool 11 and the distal end part 100a of the projecting part 100, the operator recognizes that the distal end section 8 of the inserting section 6A has reached at the removal end position CO as shown in FIG. 8. Then, the operator stops the retraction of the inserting section 6A by the foot operation using the foot switch 25 or the hand operation using the advancement/retraction switch at the main operating section 18 as described above.

In other words, the distal end section 8 of the inserting section 6A can observe a region near the distal end part of the insertion assisting tool 11 such as the rectum 502, and when the inserting section 6A reaches the removal end position CO where the inserting section 6A is removed from the large intestine, the retraction of the inserting section 6A is stopped.

Furthermore, the operator may observe the rectum 502 as needed. When the insertion assisting tool 11 includes the first and second impelling force generating members 70a and 70b, the operator can reinsert the inserting section 6A into the large intestine by advancing the large intestine 6A again, because the first and second impelling force generating members 70a and 70b are brought in contact with the rotatable barrel body 51 without fail and without idly rotating at the position.

If there is formed a gap or step between the opening 58a of the insertion assisting tool 11 and the inserting section 6A, the distal end section 8 of the inserting section 6A is stopped at the removal end position CO before entering into the opening 58a of the insertion assisting tool 11 as shown in FIG. 8.

Then, after observing the rectum 502 and the like, the operator removes the inserting section 6A from the patient via the anus 501 with insertion assisting tool 11 to end the examination of the large intestine.

At this point, the inserting section 6A is applied with the impelling force for retraction by each impelling force generating members 70a and 70 bin the guide tube fixing member 64, and is housed in the housing case main body 12 to the original looped position as shown in FIG. 41.

As described above, according to this embodiment, when the distal end part of the insertion assisting tool 11 is displayed on an endoscope image, an operator can recognize that the distal end section 8 of inserting section 6A is at the removal end position CO where the distal end section 8 can observe a region and stop the retraction of the inserting section 6A. So, the operator can observe a region such as the rectum without fail when the rectum should be observed, and also can smoothly remove the inserting section 6A when it should be removed.

In the above described examination of the large intestine for example, the inserting section 6A may produce torsion stress in advancing or retracting due to the rotation of the rotatable barrel body 51. However, in the endoscope system 1 of this embodiment, the distance between the first and second plate members 61 and 62 of the housing case main body 12 is less than twice of the maximum outer diameter at the position of the convex part included in the helical-surface shaped part 51a of the rotatable barrel body 51 which forms the outer contour of the inserting section 6A, resulting in that any curling of the inserting section 6A into a ring shape in the housing case main body 12 due to the torsion stress can be prevented.

In addition, each of the guide tubes 13 and 14 has an inner diameter which is less than twice of the maximum outer diameter at the position of the convex part included in the helical-surface shaped part 51a of the rotatable barrel body 51, resulting in that any curling of the inserting section 6A into a ring shape in the housing case main body 12 due to the torsion stress can be prevented. Therefore, the inserting section 6A can smoothly rotate about the axis in the housing case main body 12 and in the guide tube toward the distal end 13.

Also, an operator can visually recognize the movement of the inserting section 6A, especially the rotation of the rotatable barrel body 51, because the housing case main body 12 and each of the guide tubes 13 and 14 are formed of a transparent or semitransparent material.

The connections between the insertion assisting tool 11, the guide tube toward the distal end 13, the housing case main body 12, and the guide tube toward operating section 14 are individually kept watertight, resulting in that any scattering of liquids such as drainage in the large intestine in an operation room can be prevented. So, the inserting section with a case 6 has an extremely hygienic structure.

The first and second impelling force generating members 70a and 70b arranged in the assisting tool 11 causes the rotatable barrel body 51 of the endoscope system 1 to produce impelling force, and enhances the ease of introduction and insertion of the rotatable barrel body 51 into a body cavity, the large intestine in this embodiment. Since the guide tube fixing member 64 is provided with the two first and second impelling force generating members 70a and 70b, the rotatable barrel body 51 is positioned in the guide tube fixing member 64 in the longitudinal direction in a stable way, which allows impelling force to be effectively produced for advancing the rotatable barrel body 51 under the uniform pressure from the first and second impelling force generating members 70a and 70b.

As described above, the rotatable self-propelling endoscope 2 and its inserting section with a case 6 of this embodiment have an excellent structure which enables the inserting section 6A which is not inserted into a region to be examined yet to be smoothly inserted into the region to be examined, and provides operability.

### Second Embodiment

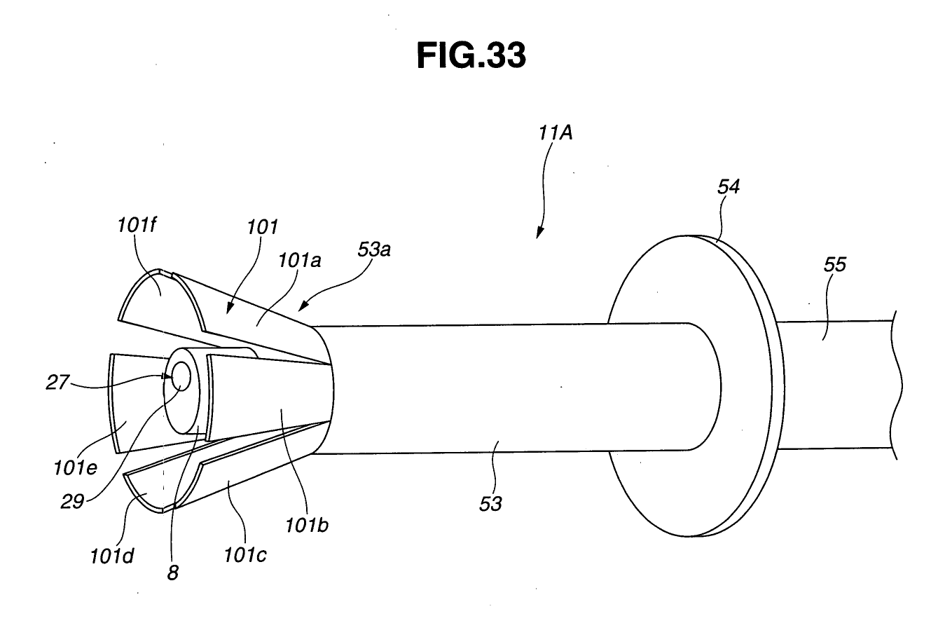

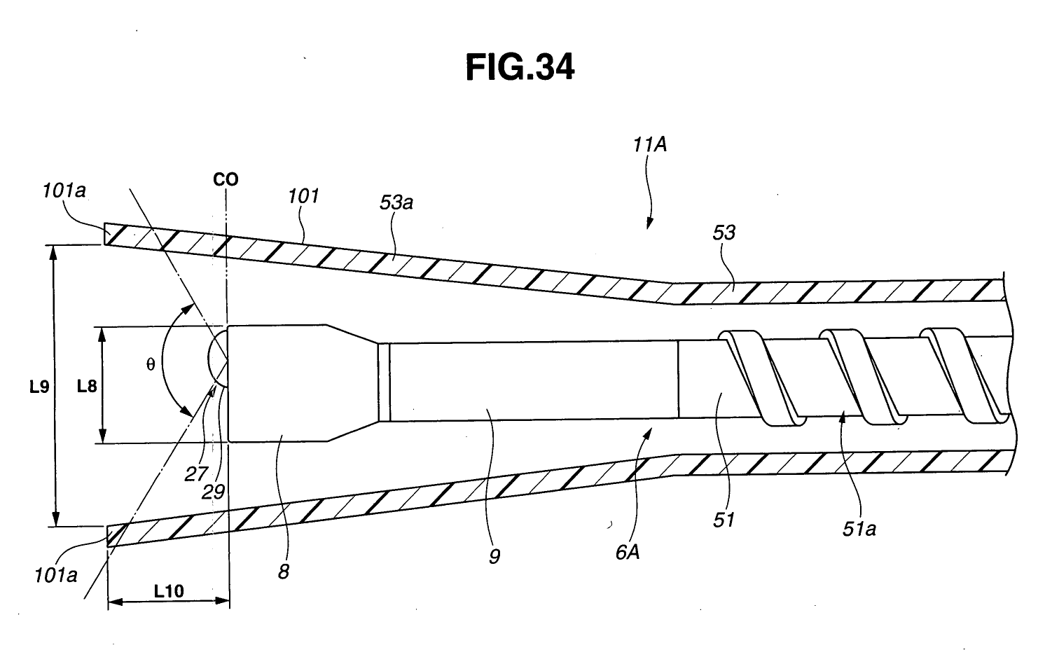

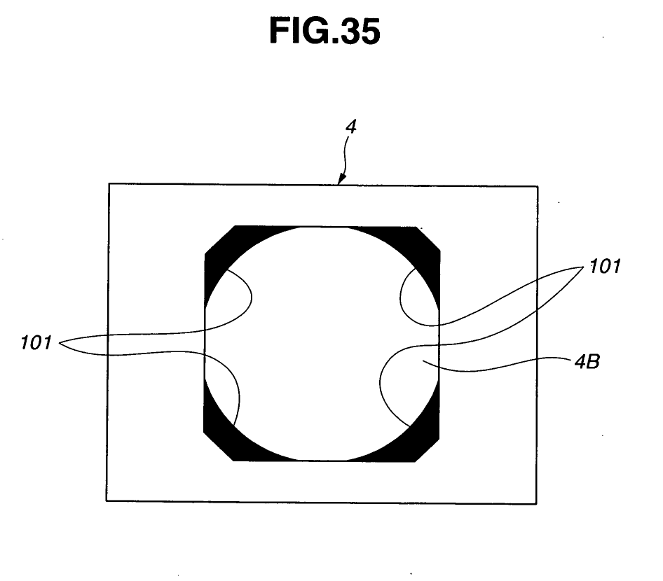

FIG. 33 to FIG. 35 are diagrams showing a second embodiment of the present invention: FIG. 33 is a perspective diagram showing a structure of an insertion assisting tool through which an inserting section is inserted; FIG. 34 is a cross sectional diagram showing the insertion assisting tool of FIG. 33; and FIG. 35 is a diagram showing an example of a monitor display when an inserting section reaches at the removal end position shown in FIG. 34.

In FIG. 33 to FIG. 35, the components similar to those of the first embodiment are designated by the same reference numerals and the explanation of these components will be omitted in the following description, and only the differences will be explained below.

In the second embodiment, the projecting part 100 of the insertion assisting tool 11A has an improved structure.

Specifically, as shown in FIG. 33, the distal end insertion tube 53a of the insertion assisting tool 11A has a projecting part 101. The projecting part 101 constitutes a tapered barrel-shaped member which is formed by extending the entire outer circumferential part of the insertion tube 53a to be flared toward the opening of the insertion assisting tool 11A.

The barrel-shaped member 101 is configured to have a plurality of divided sides 101a to 101f in an insertion direction and notches between the plurality of sides 101a to 101f.

The barrel-shaped member 101 is only required to have at least one side as a projecting part as in the case of the projecting part 100 of the first embodiment.

The barrel-shaped member 101 is formed of a flexible synthetic resin such as silicon, similarly to the first embodiment.

The insertion assisting tool 11A of such a structure is, as shown in FIG. 34, similar to the first embodiment, is configured to allow the inserting section 6A to be retracted to the removal end position Co where an image of the distal end part 101a of the barrel-shaped member 101 and a part near the distal end part 101a can be picked up, in retracting the inserting section 6A.

In this case, the removal end position CO is desirably set to be located at a position which meets the following sizes: a viewing angle θ is 120 degrees, an L8 is 15 mm, an L9 is 35 mm, and an L10 is 150 to 160 mm, where the L8 is an outer diameter of the distal end section 8, L10 is the length between the distal end part 101a of the barrel-shaped member 101 to the distal end surface of the distal end section 8, L9 is the opening diameter of the barrel-shaped member 101, and the viewing angle θ is the viewing angle of the image pickup unit 27 in the distal end section 8. These sizes may be applied to the first embodiment.

The other structures are similar to those of the first embodiment.

In this embodiment, the rotatable self-propelling endoscope 2 is operated in the same way as in the first embodiment. In other words, in retracting the inserting section 6A to remove it from a body cavity such as the large intestine, an operator causes the monitor 4 to display an endoscope image based on an image pickup signal captured by the image pickup unit 27. In this state, when the barrel-shaped member 101 (including the distal end part 101a) which is the projecting part of the distal end part of the insertion assisting tool 11A is displayed in the endoscope image, the operator determines that the distal end section 8 of the inserting section 6A has reached the removal end position CO. An example of the screen display on the monitor 4 is shown in FIG. 35.

That is, as shown in FIG. 35, when the endoscope image 4B displayed on the monitor 4 includes the barrel-shaped member 101 of the insertion assisting tool 1A and the distal end part 1010a of the barrel-shaped member 101, the operator can recognize that the distal end section 8 of the inserting section 6A has reached at the removal end position CO as shown in FIG. 35. Then, the operator stops the retraction of the inserting section 6A by the foot operation using the foot switch 25 or the hand operation using the advancement/retraction switch at the main operating section 18 as in the case of the first embodiment.

In other words, the distal end section 8 of the inserting section 6A can observe a region near the distal end part of the insertion assisting tool 11 such as the rectum 502, and when the inserting section 6A reaches the removal end position CO where the inserting section 6A is removed from the large intestine, the retraction of the inserting section 6A is stopped.

So, according to this embodiment, the operator can observe a region such as the rectum without fail when the rectum should be observed, and also can smoothly remove the inserting section 6A when it should be removed.

Since the barrel-shaped member 101 has the plurality of sides 101a to 101f and the notches between the sides 101a to 101f (see FIG. 33), an advantage can be provided that the barrel-shaped member 101 can be inserted by following an enteric canal in the large intestine more easily compared to the one which is configured to have an outer circumferential part as only one tapered element. The other effects are similar to those of the first embodiment.

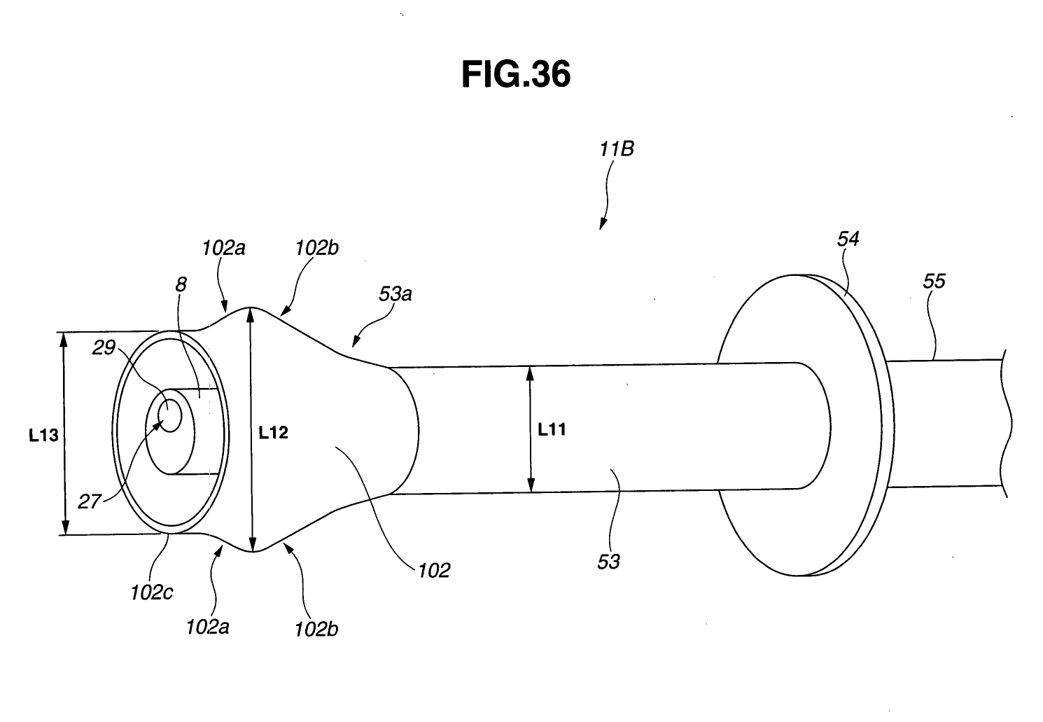

The barrel-shaped member 101 of this embodiment may be modified as shown in FIG. 36.

The modified example of the barrel-shaped member 101 will be explained below with reference to FIG. 36.

### Modified Example 1

FIG. 36 is a perspective diagram showing a modified example of the cylinder-shaped member of the second embodiment, and shows a structure of an insertion assisting tool through which an inserting section is inserted. In FIG. 36, the components similar to those of the first and second embodiments are designated by the same reference numerals and the explanation of these components will be omitted in the following description, and only the differences will be explained below.

In this modified example 1, the barrel-shaped member 101 of the insertion assisting tool 11A of the second embodiment is improved.

Specifically, as shown in FIG. 36, the barrel-shaped member 102 of the insertion assisting tool 11B is formed in a barrel shape which is generally the same as that of the second embodiment (see FIG. 35), but has a tapered proximal end, and has an outer circumferential surface toward the opening which partly projects outward in an arc shape. Therefore, slightly curved arc-shaped parts 102a and 102b are formed toward the opening and the proximal end.

The barrel-shaped member 102 is also configured to have a maximum outer diameter L12 at the projected part between the two arc-shaped parts 102a and 102b.

The insertion assisting tool 11B is configured to have a diameter L13 at the distal end of the barrel-shaped member 102 which is smaller than the maximum outer diameter L12 of the barrel-shaped member 102 and also is larger than the outer diameter L11 of the insertion tube 53 (see FIG. 36).

The positions of the arc-shaped parts 102a and 102b are not limited to those shown in FIG. 36, and may be offset in the direction of the insertion axis of the barrel-shaped member 102 as needed.

Furthermore, the barrel-shaped member 102 is formed of a flexible synthetic resin such as silicon as in the case of the second embodiment.

The other structures are similar to those of the first and second embodiments.

In the modified example 1, the same operations are provided as in the case of the second embodiment. In other words, in retracting the inserting section 6A to remove it from a body cavity such as the large intestine, an operator causes the monitor 4 to display an endoscope image based on an image pickup signal captured by the image pickup unit 27. In this state, when the barrel-shaped member 102 (including the distal end part 102a) at the distal end part of the insertion assisting tool 11A is displayed in the endoscope image, the operator determines that the distal end section 8 of the inserting section 6A has reached the removal end position CO.

Then, the operator stops the retraction of the inserting section 6A by the foot operation using the foot switch 25 or the hand operation using the advancement/retraction switch at the main operating section 18 as in the case of the second embodiment.

In other words, the distal end section 8 of the inserting section 6A can observe a region near the distal end part of the insertion assisting tool 11 such as the rectum 502, and when the inserting section 6A reaches the removal end position CO where the inserting section 6A is removed from the large intestine, the retraction of the inserting section 6A is stopped.

So, according to this modified example 2, the same effects can be obtained as in the case of the second embodiment, and also the barrel-shaped member 102 having the arc-shaped 102a and 102b can be easily inserted into a body cavity such as anus.

### Third Embodiment

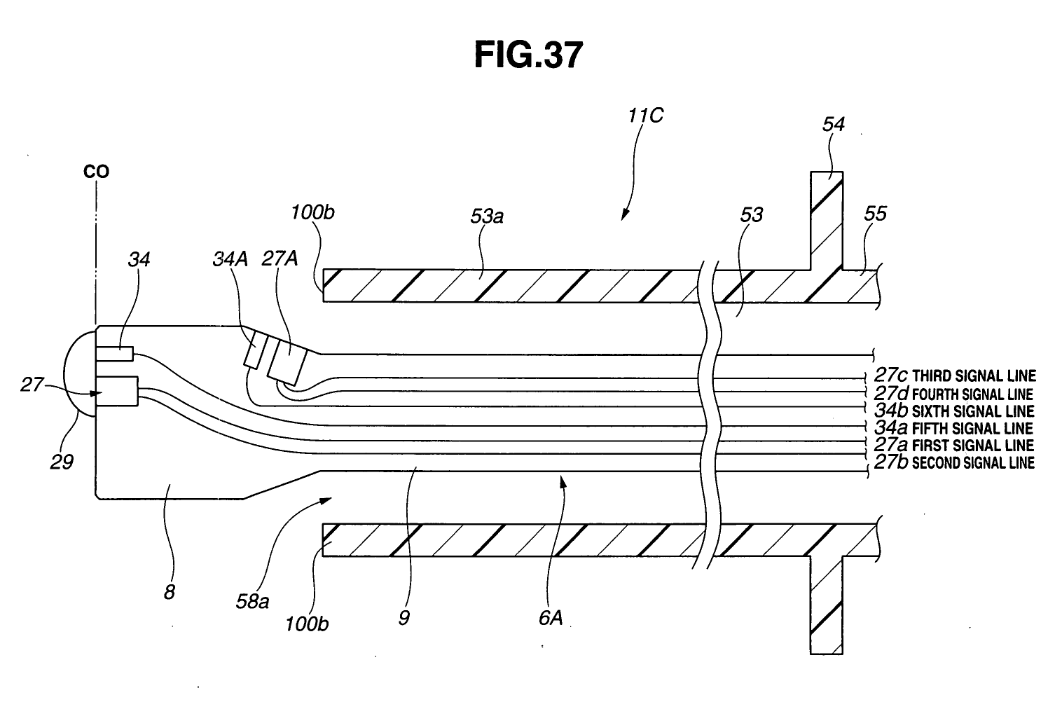

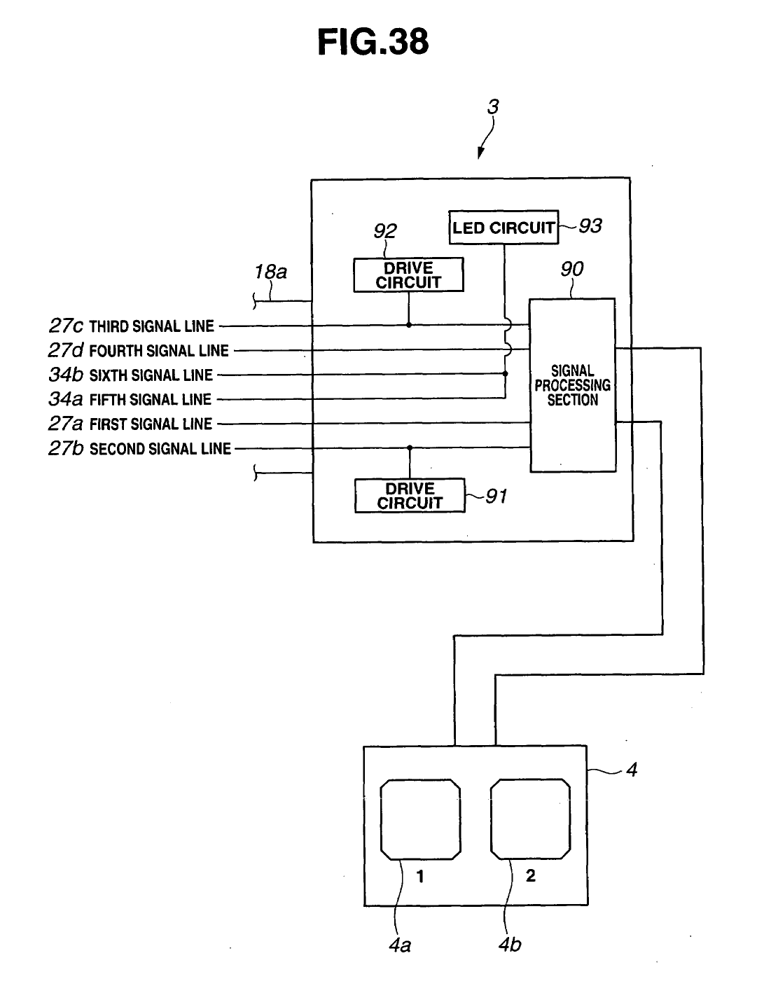

FIG. 37 and FIG. 38 are diagrams showing a third embodiment of the present invention: FIG. 37 is a cross sectional diagram showing a structure of an insertion section which is inserted through an insertion assisting tool; and FIG. 38 is a block diagram showing an electrical structure of the control device 3 which is electrically connected to an inserting section. In FIG. 37 and FIG. 38, the components similar to those of the first embodiment are designated by the same reference numerals and the explanation of these components will be omitted in the following description, and only the differences will be explained below.

In this third embodiment, a second image pickup unit 27A is provided at the proximal end of the distal end section 8 of the inserting section 6A, so that when an endoscope image which is captured by the image pickup unit 27A and is displayed on the monitor 4 includes the distal end part 100b of the insertion assisting tool 11, an operator can recognize that the distal end section 8 has reached the removal end position CO.

Specifically, as shown in FIG. 37, at the distal end section 8 of the inserting section 6A, in addition to the image pickup unit 27 of the first embodiment and a plurality of LEDs 34, a second image pickup unit 27A and a second plurality of LEDs 34A for capturing an image of the distal end part 100b of the insertion assisting tool 11C are provided at the proximal end part thereof.

The second image pickup unit 27A generally has the same structure as that of the image pickup unit 27 of the first embodiment, and has a second image pickup device (not shown) such as a CCD, objective lenses, and the like arranged therein. The second plurality of LEDs 34A also generally have the same structures as those of the plurality of LEDs 34 of the first embodiment.

The image pickup unit 27 is provided with two, first and second, signal lines 27a and 27b which extend therefrom. The first signal line 27a is electrically connected to a signal processing section 90 in the control device 3 shown in FIG. 38, while the second signal line 27b is electrically connected to a drive circuit 91 and a signal processing section 90 in the control device 3 shown in FIG. 38.

The second image pickup unit 27A is provided with two, third and fourth, signal lines 27c and 27d which extend therefrom. The third signal line 27c is electrically connected to a drive circuit 92 and the signal processing section 90 in the control device 3 shown in FIG. 38, while the fourth signal line 27d is electrically connected to the signal processing section 90 in the control device 3 shown in FIG. 38.

The LED 34 is provided with a fifth signal line 34a which extends therefrom. The fifth signal line 34a is electrically connected to an LED circuit 93 in the control device 3 shown in FIG. 38.

The other LED 34A is provided with a sixth signal line 34b which extends therefrom. The sixth signal line 34b is electrically connected to the LED circuit 93 in the control device 3 shown in FIG. 38.

Unlike the first and second embodiments, the insertion assisting tool 11C is configured not to have a projecting part, and has a typical structure which has been conventionally used.

The positions of the second image pickup unit 27A and LED 34A are not limited to the those at the proximal end part of the distal end section 8, and may be located at any position where an image of the distal end part 100b of the insertion assisting tool 11C can be picked up so that an operator can recognize that distal end section 8 has reached the removal end position CO.

Next, a schematic structure of the control device 3 to which the first to sixth signal lines 27a to 34b are connected will be explained below with reference to FIG. 38.

As shown in FIG. 38, the control device 3 has two drive circuits 91 and 92, the signal processing section 90 which is electrically connected the monitor 4, and the LED circuit 93.

The drive circuit 91 generates driving signals for driving the image pickup device 31 such as a CCD in the image pickup unit 27, and supplies the signals via the second signal line 27b to the image pickup device 31 to drive the image pickup device 31.

Then the driven image pickup device 31 picks up an image pickup signal, which is output by the image pickup unit 27 via the first signal line 27a to the signal processing section 90.

The drive circuit 92 generates driving signals for driving an image pickup device (not shown) such as a CCD in the image pickup unit 27A, and supplies the signals via the third signal line 27c to the image pickup device (not shown) to drive the image pickup device.

Then the driven image pickup device (not shown) picks up an image pickup signal, which is output by the image pickup unit 27A via the fourth signal line 27d to the signal processing section 90.

The LED circuit 93 generates driving signals for turning on the LEDs 34 and 34A, and supplies the signals via the fifth and sixth signal lines 34a and 34b to the LEDs 34 and 34A to turn them on, respectively.

The signal processing section 80 is configured to process the image pickup signals supplied from the image pickup units 27 and 27A to convert them into picture signals, and individually output the converted picture signals to the monitor 4.

The monitor 4 is provided with two input terminal systems for inputting the two picture signals, and is able to display the two endoscope images based on each of the input picture signals in one screen (a first screen 4a and a second screen 4b).

For example, the first screen 4a of the monitor 4 displays an image captured by the second image pickup unit 27A for checking that the inserting section 6A has reached the removal end position CO, while the second screen 4b displays a normal endoscope image captured by the image pickup unit 27. However, the way to display images is not limited to this, and the images may be switched to be displayed on the first screen 4a and the second screen 4b, respectively.

In the above description, the signal processing section 90 individually outputs the picture signals based on the image pickup signals from the image pickup units 27 and 27A to the monitor 4, but the two signals may be processed to be superimposed to each other to generate a superimposed picture signal for a two-screen display on the monitor 4.

The other structures are similar to those of the first embodiment.

In this embodiment, the same operations are provided as those of the first embodiment, but when the inserting section 6A is retracted to remove it from a body cavity such as the large intestine, the first screen 4a of the monitor 4 displays a picture seen from the rear part of the distal end section 8 of the inserting section 6A which is captured by the second image pickup unit 27A.

An operator causes the inserting section 6A to be retracted while watching the first screen 4a of the monitor 4. In this case, the operator determines that the distal end section 8 of the inserting section 6A has reached the removal end position CO when the picture on the first screen 4a includes the distal end part 100b of the insertion assisting tool 11C.

Then, the operator stops the retraction of the inserting section 6A by the foot operation using the foot switch 25 or the hand operation using the advancement/retraction switch at the main operating section 18 as in the case of the first embodiment.

In other words, the distal end section 8 of the inserting section 6A can observe a region near the distal end part of the insertion assisting tool 11A such as the rectum 502, and when the inserting section 6A reaches the removal end position CO where the inserting section 6A is removed from the large intestine, the retraction of the inserting section 6A is stopped.

The other operations are similar to those of the first embodiment.

Therefore, according to this embodiment, the same effects can be obtained as in the case of the first embodiment even when the insertion assisting tool 11C having a normal configuration is used, by providing the second image pickup unit 27A and the LED 34A to the distal end section 8 toward the proximal end thereof. Furthermore, the second image pickup unit 27A and the LED 34A at the distal end section 8 toward the proximal end thereof enables an observation for a different field of view together with a normal observation, which provides an effect in enhancing the observation property.

### Fourth Embodiment

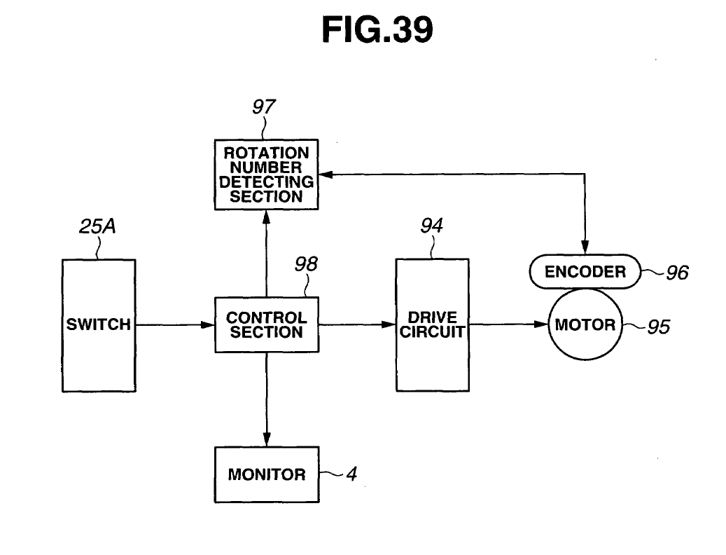

FIG. 39 is a block diagram showing a fourth embodiment of the present invention, and shows a structure of a main component circuit block for controlling a retraction of an inserting section.

In this fourth embodiment, the retraction of the inserting section 6A is electrically controlled so that the distal end section 8 of the inserting section 6A is located at the removal end position CO.

Specifically, as shown in FIG. 39, the main component circuit block of this embodiment has a switch 25A which constitutes the foot switch 25 or the advancement/retraction switch in the main operating section 18, a controlling section 98 in the control device 3, a drive circuit 94, a motor 95, and an encoder 96 which are provided in the motor box 16, a rotation number detecting section 97 in the control device 3, and the monitor 4.

The encoder 96 is provided to a rotary shaft of the motor 95 to detect a rotation number of the motor 95, and supplies the detected result to the rotation number detecting section 97.

The rotation number detecting section 97 detects a rotation number on the basis of a pitch of the convex and concave parts of the helical-surface shaped part 51a formed on the rotatable barrel body 51 based on the detection result from the encoder 96 (which is also called as number of pitch rotation), and outputs the detected result to the controlling section 98.

The controlling section 98 generates a controlling signal for controlling the motor 95, and supplied it to the drive circuit 94. And the drive circuit 94 converts the controlling signal from the controlling section 98 into a driving signal for driving, and supplies it to the motor 95 to control the rotation of the motor 95.

The controlling section 98 also has a counting section for counting the detection result (the number of pitch rotation) from the rotation number detecting section 97 (not shown), so that the controlling section 98 generates the controlling signal based on the counted value which is counted by the counting section.

That is, in advancing the inserting section 6A, a pitch rotation number value of the convex and concave parts of the helical-surface shaped part 51a which is counted corresponding to the advanced distance is calculated. In retracting the inserting section 6A, the rotation of the motor 95 is controlled by the controlling section 98 so that the inserting section 6A is caused to be retracted by the value of the calculated pitch rotation number.

So, in advance, the counting section (not shown) in the controlling section 98 can be reset when the distal end section 8 of the inserting section 6A is at the removal end position CO, so that the controlling section 98 can control the distal end section 8 to be located at the removal end position CO in retracting, by controlling the rotation of the motor 95 based on the pitch rotation number value.

In other words, the controlling section 98 will control the drive circuit 94 to cause the motor 95 to be stopped after the rotation control of the motor 95 based on the pitch rotation number value.

So, in this embodiment, the controlling section 98 allows the retraction of the inserting section 6A to be stopped while the distal end section 8 is located at the removal end position CO.

The other structures and operations are similar to those of the first embodiment.

Therefore, according to this embodiment, even when the motor 95 is electrically controlled, the retraction of the inserting section 6A can be automatically stopped so that the distal end section 8 of the inserting section 6A is automatically located at the removal end position CO.

A rotation number in advancing and a rotation number in retracting may be displayed on a monitor in order to stop the rotation of the motor at the point when the two rotation numbers are identical, so that the distal end section 8 can be located at the removal end position CO.

### Fifth Embodiment

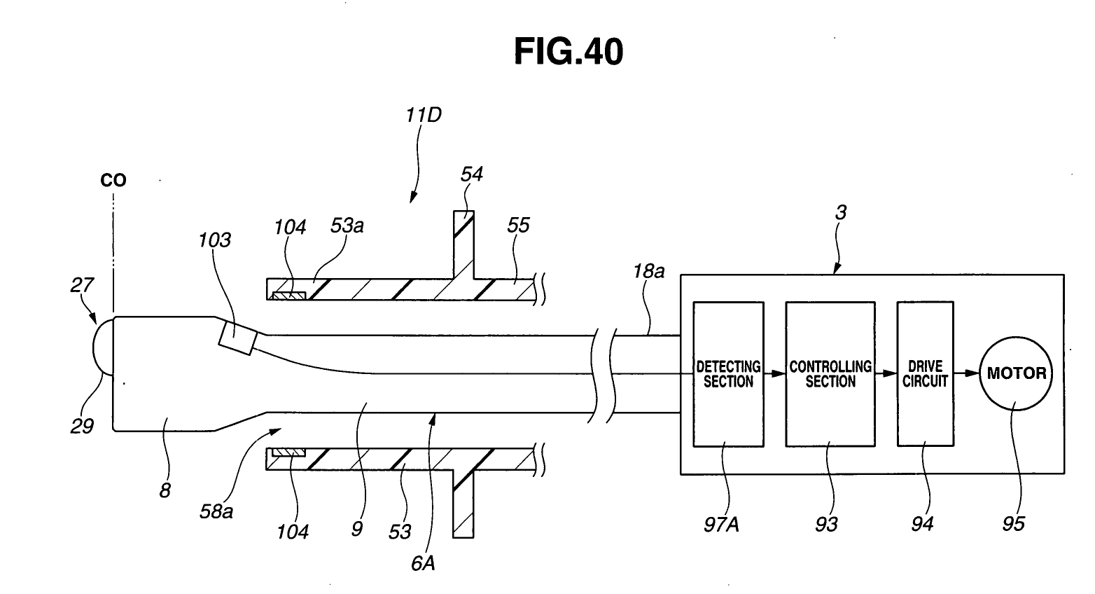

FIG. 40 is a structure diagram showing a fifth embodiment of the present invention, and shows an insertion assisting tool through which an inserting section is inserted, and a structure of a main component circuit block in a controlling device which is electrically connected to the insertion assisting tool. In FIG. 40, the components similar to those of the first and fourth embodiments are designated by the same reference numerals and the explanation of these components will be omitted in the following description, and only the differences will be explained below.

In this fifth embodiment, a reflection type sensor 103 is provided at the distal end section 8 of the inserting section 6A at a position close to the proximal end thereof, so that when the reflection type sensor 103 detects the proximal end part of the insertion assisting tool 11D, an operator can recognized that the distal end section 8 has reached the removal end position CO.

Specifically, as shown in FIG. 40, the reflection type sensor 103 is provided at the proximal end part of the distal end section 8 of the inserting section 6A. The reflection type sensor 103 may be constituted of a photo-reflector.

The reflection type sensor 103 detects the proximal end part of the insertion assisting tool 11D and outputs a detecting signal in the retraction of the inserting section 6A. The reflection type sensor 103 is electrically connected a detecting section 97A in the control device 3 via a signal line to output the detecting signal to the detecting section 97A.

Meanwhile, the control device 3 which constitutes the main structure block of this embodiment has the detecting section 97A to which a detecting signal is supplied from the reflection type sensor 103, the controlling section 98, and the drive circuit 94 and the motor 95 which are provided in the motor box 16.

The detecting section 97A detects a detecting signal supplied from the reflection type sensor 103, and outputs a timing signal as a result of the detection to the controlling section 98.

The controlling section 98 generates a controlling signal for controlling the motor 95, and supplies the signal to the drive circuit 94. Then the drive circuit 94 converts the controlling signal from the controlling section 98 into a driving signal for driving, and supplies it to the motor 95 to control the rotation of the motor 95.

Upon the supply of the timing signal from the detecting section 97A, the controlling section 98 determines that the distal end section 8 has reached the removal end position CO, and controls the drive circuit 94 to stop the rotation of the motor 95.

That is, when the reflection type sensor 103 detects the distal end part of the insertion assisting tool 11D, the controlling section 98 determines that the distal end section 8 has reached the removal end position CO.

So, the retraction of the inserting section 6A can be automatically stopped while the distal end section 8 is located at the removal end position CO.

Therefore, according to this embodiment, as in the case of the fourth embodiment, the retraction of the inserting section 6A can be automatically stopped so that the distal end section 8 of the inserting section 6A is automatically located at the removal end position CO. The rotation of the motor is stopped by an operator when the monitor 4 displays that the reflection type sensor 103 detects the distal end part of the insertion assisting tool 11D and the operator checks the display.

In this embodiment, when an optical sensor is used instead of the reflection type sensor 103, similar operations and effects can be obtained by providing a light source 104 such as an LED at the inner circumferential surface at the distal end of the insertion assisting tool 11D or inside of the distal end part as shown in FIG. 40.

### Sixth Embodiment

FIG. 41 shows a sixth embodiment of the present invention, and is a top view showing an inserting section seen from the top thereof. In FIG. 41, the components similar to those of the first embodiment are designated by the same reference numerals and the explanation of these components will be omitted in the following description, and only the differences will be explained below.

In the sixth embodiment, an identification mark 106 which shows a removal end position is provided at a predetermined position on the inserting section 6A inserted through the transparent or semitransparent guide tube toward the distal end 13.

Specifically, as shown in FIG. 41, the inserting section with a case 6 of this embodiment generally has the same structure as that of the first embodiment, and is provided with the identification mark 106 at a predetermined position on the inserting section 6A which constitutes the inserting section with a case 6.

The identification mark 106 may be of any type which an operator can visually identify. For example, the identification mark 106 may be designed to be identified by an index or a color.

The predetermined position may be, for example, a position which can be viewed from outside of the guide tube toward the distal end 13 near the connection between the guide tube toward the distal end 13 and the insertion assisting tool 11, when the distal end section 8 of the inserting section 6A is located at the removal end position CO relative to the insertion assisting tool 11 in advance (see FIG. 8).

Therefore, the identification mark 106 at the predetermined position on the inserting section 6A allows an operator to retract the inserting section 6A, watching the state of the retracting inserting section 6A through the guide tube toward the distal end 13.

And when the operator views the identification mark 106 on the inserting section 6A through the guide tube toward the distal end 13, the operator recognizes that distal end section 8 has reached the removal end position CO, and performs the operations similar to those in the first embodiment to stop the retraction of the inserting section 6A.

Instead of the above position, the identification mark 106 may be located, for example, at a predetermined position on the inserted inserting section 6A in the housing case main body 12 which is configured with the transparent or semitransparent first and second plate members 61, 62. In this case, other plurality of identification marks 106 which are separated by predetermined spaces may be provided following the identification mark 106 on the inserting section 6A, which allows an operator to recognize at a glance the length of the inserting section 6A which has been inserted into a body cavity.

Therefore, according to this embodiment, the same effects can be obtained as in the case of the first embodiment, and also the identification mark 106 at the predetermined position on the inserting section 6A allows an operator to recognize that the distal end section 8 has reached the removal end position Co.

### Seventh Embodiment

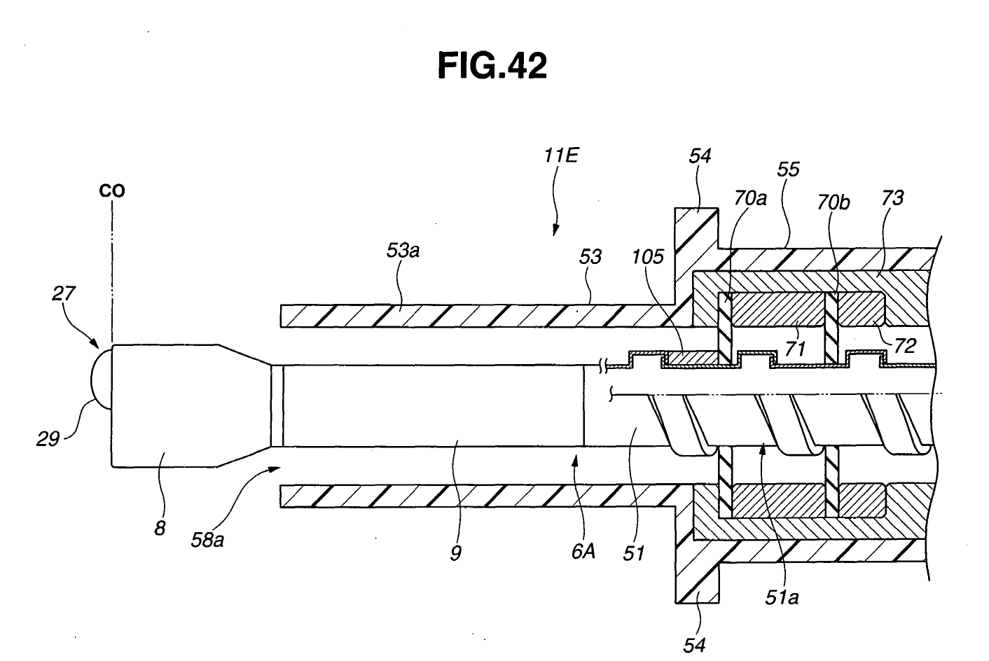

FIG. 42 shows a seventh embodiment of the present invention, and is a cross sectional diagram showing a distal end part of an insertion assisting tool to which an impelling force generating member is provided. In FIG. 42, the components similar to those of the first embodiment are designated by the same reference numerals and the explanation of these components will be omitted in the following description, and only the differences will be explained below.

In this embodiment, the inserting section with a case 6 generally has the same structure as that of the first embodiment shown in FIG. 27, but a locking member 105 is provided between the convex and concave parts of the rotatable barrel body 51 which contacts the first impelling force generating member 70a so that the distal end section 8 of the inserting section 6A is located at the removal end position Co.

Specifically, as shown in FIG. 42, the structures of the insertion assisting tool 11, and the first and second impelling force generating members 70a and 70b are generally the same as those shown in FIG. 27, and so will not be explained below. However, the insertion assisting tool 11 does not have a projecting member 100 at the distal end part thereof.

The rotatable barrel body 51 in the inserting section 6A of this embodiment is provided with a locking member 105 as described above. The locking member 105 functions to stop the retraction of the inserting section 6A and also to locate the distal end section 8 of the inserting section 6A at the removal end position CO when the locking member 105 contacts the distal end side of the first impelling force generating member 70a, for example.

The locking member 105 may be formed by fixing a synthetic resin such as silicon at the position between the convex and concave parts of the corresponding helical-surface shaped part 51a, or by fixing a member such as stainless steel, as in the case of the rotatable barrel body 51, at the corresponding position by welding.

The other structures are similar to those of the first embodiment.

In this embodiment, since the locking member 105 is provided at a predetermined position on the rotatable barrel body 51 in the inserting section 6A, when the inserting section 6A is retracted, when the locking member 105 in the rotatable barrel body 51 is brought in contact with the first impelling force generating member 70b, the retraction of the inserting section 6A is automatically stopped so that the inserting section 6A is not retracted beyond the removal complete position CO.

The inserting section 6A can be, as in the case of the first embodiment, reinserted into a body cavity from the removal complete position Co.

However, in this embodiment, the locking member 105 provided at the rotatable barrel body 51 makes it difficult to insert the distal end section 8 of the inserting section 6A through the inside of the insertion assisting tool 11, and so the inserting section 6A needs to be removed with the insertion assisting tool 11 from a body cavity with the inserting section 6A being at the removal end position CO.

The other operations are generally similar to those of the first embodiment.

Therefore, according to this embodiment, the same effects can be obtained as in the case of the first embodiment, and also the retraction of the inserting section 6A can be automatically stopped with the inserting section 6A being at the removal end position CO, when the insertion assisting tool 11 is provided with the first and second impelling force generating member 70a, 70b.

The locking member 105 may be located at a position on rotatable barrel body 51 depending on the positions where the first and second impelling force generating member 70a, 70b are provided.

The present invention which has been described by way of the above embodiments is not limited to the embodiments and the modified examples, and various modifications can be made to the above described embodiments in practicing without departing from the scope of the present invention. In addition, since the above described embodiments include some inventions at different stages, a plurality of constituent features disclosed herein may be conveniently combined to create different inventions.

For example, if the above described problems which the present invention was made to resolve can be resolved and also the above described effects can be obtained even when some constituent features are omitted from all of the constituent features included in each embodiment, the structure without the omitted constituent features may create an invention.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

### APPENDIX

### Appendix 1

An endoscope system, comprising:

an inserting section, including,
a distal end rigid section which has a first image pickup section,
an inserting section main body which has a distal end part provided with the distal end rigid section and is flexible to be inserted into a body cavity, and
a rotatable barrel body which is rotatably fitted on the inserting section main body and has a helical-surface shaped part with a helical convex-concave structure; and

a housing case, including,
an inserting section housing case main body for housing the inserting section, and
an insertion assisting tool through which the inserting section derived from the housing case main body is inserted to be guided into the body cavity, and

the endoscope system further comprising: in order to retract the inserting section,

an endoscope inserting section with a case which is configured so that the inserting section is retracted to a position where an image of the distal end section can be picked up by the insertion assisting tool;

a controlling device for controlling the rotation of the rotatable barrel body; and

a rotation number detecting section for detecting the rotation of the rotatable barrel body.

### Appendix 2

The endoscope system according to the appendix 1, wherein

a sensor for detecting a complete removal is provided at the proximal end of the distal end part of the inserting section.

What is claimed is:

1. An endoscope inserting section with a case, comprising:
an inserting section, including,
a distal end rigid section which has a first image pickup section,
an inserting section main body which has a distal end part provided with the distal end rigid section and is flexible to be inserted into a body cavity, and
a rotatable barrel body which is rotatably fitted on the inserting section main body and has a helical-surface shaped part with a helical convex-concave structure; and
a housing case, including,
an inserting section housing case main body for housing the inserting section, and
an insertion assisting tool through which the inserting section derived from the housing case main body is inserted to be guided into the body cavity, wherein
the insertion assisting tool is configured to retract the inserting section to a position where an image of the distal end part of the insertion assisting tool can be picked up, when the inserting section is retracted.

2. The endoscope inserting section with a case according to claim 1, wherein
the insertion assisting tool is able to cause the inserting section to be retracted to a position where an image of an inner circumferential surface of the insertion assisting tool can be picked up.

3. The endoscope inserting section with a case according to claim 1, wherein
the insertion assisting tool has at the distal end part provided with a projecting part which projects in the inserting direction of the insertion assisting tool.

4. The endoscope inserting section with a case according to claim 3, wherein
the projecting part is a projected cylindrical member with an arc-shaped distal end part, the projecting part being formed by extending a part of the distal end part of the insertion assisting tool in the inserting direction of the insertion assisting tool.

5. The endoscope inserting section with a case according to claim 3, wherein
the projecting part is a tapered barrel-shaped member which is formed by extending the entire or a part of the outer circumferential part at the distal end part of the insertion assisting tool to be flared toward the opening of the insertion assisting tool.

6. The endoscope inserting section with a case according to claim 5, wherein
the barrel-shaped member is configured to include a plurality of parts which are divided in the inserted direction of the insertion assisting tool and notches between the plurality of parts.

7. The endoscope inserting section with a case according to claim 3, wherein
the projecting part is formed of a flexible member.

8. The endoscope inserting section with a case according to claim 4, wherein
the projecting part is formed of a flexible member.

9. The endoscope inserting section with a case according to claim 5, wherein
the projecting part is formed of a flexible member.

10. The endoscope inserting section with a case according to claim 6, wherein
the projecting part is formed of a flexible member.

11. The endoscope inserting section with a case according to claim 3, wherein
the outer diameter of the projecting part is larger than the outer diameter of the insertion assisting tool.

12. The endoscope inserting section with a case according to claim 4, wherein
the outer diameter of the projecting part is larger than the outer diameter of the insertion assisting tool.

13. The endoscope inserting section with a case according to claim 5, wherein
the outer diameter of the projecting part is larger than the outer diameter of the insertion assisting tool.

14. The endoscope inserting section with a case according to claim 6, wherein
the outer diameter of the projecting part is larger than the outer diameter of the insertion assisting tool.

15. The endoscope inserting section with a case according to claim 7, wherein
the outer diameter of the projecting part is larger than the outer diameter of the insertion assisting tool.

16. The endoscope inserting section with a case according to claim 1, wherein
the insertion assisting tool has an impelling force generating section for causing the rotatable barrel body to generate an impelling force.

17. The endoscope inserting section with a case according to claim 2, wherein
the insertion assisting tool has an impelling force generating section for causing the rotatable barrel body to generate an impelling force.

18. The endoscope inserting section with a case according to claim 3, wherein
the insertion assisting tool has an impelling force generating section for causing the rotatable barrel body to generate an impelling force.

19. The endoscope inserting section with a case according to claim 4, wherein
the insertion assisting tool has an impelling force generating section for causing the rotatable barrel body to generate an impelling force.

20. The endoscope inserting section with a case according to claim 5, wherein
the insertion assisting tool has an impelling force generating section for causing the rotatable barrel body to generate an impelling force.

21. The endoscope inserting section with a case according to claim 6, wherein
the insertion assisting tool has an impelling force generating section for causing the rotatable barrel body to generate an impelling force.

22. The endoscope inserting section with a case according to claim 7, wherein
the insertion assisting tool has an impelling force generating section for causing the rotatable barrel body to generate an impelling force.

23. The endoscope inserting section with a case according to claim 11, wherein
the insertion assisting tool has an impelling force generating section for causing the rotatable barrel body to generate an impelling force.

24. The endoscope inserting section with a case according to claim 1, wherein
the inserting section has a second image pickup section at the distal end section.

25. The endoscope inserting section with a case according to claim 1, wherein
the inserting section has an identification mark at a predetermined position.

26. The endoscope inserting section with a case according to claim 1, wherein
the rotatable barrel body has a locking member between the convex and concave parts.

27. A method for removing an endoscope inserting section, which is a method for removing an inserting section having a flexible inserting section main body, a rotatable barrel body that is rotatably fitted on the inserting section main body to make the inserting section main body self-propelled through a body cavity, a distal end rigid section having an image pickup section, and a bending section, from the body cavity, comprising:
rotating the rotatable barrel body so that the inserting section is retracted, and housing a predetermined part of the inserting section into an inner bore of the insertion assisting tool which is inserted in the body cavity;
stopping the rotation of the rotatable barrel body; and
removing the inserting section from the body cavity with the insertion assisting tool.

28. The method for removing an endoscope inserting section according to claim 27, comprising:
checking that the distal end part of the inserting section has reached a predetermined position during the removal,
stopping the rotation of the rotatable barrel body; and
removing the inserting section from the body cavity with the insertion assisting tool.

29. The method for removing an endoscope inserting section according to claim 27, comprising:
rotating the rotatable barrel body so that the inserting section is retracted;
checking that the predetermined part of the inserting section is housed into the inner bore of the insertion assisting tool which is inserted in the body cavity, by using a monitor which displays an endoscope image;
stopping the rotation of the rotatable barrel body; and
removing the inserting section from the body cavity with the insertion assisting tool.

30. The method for removing an endoscope inserting section according to claim 27, further comprising:
checking that the predetermined part of the inserting section is housed into the inner bore of the insertion assisting tool, by using an image of a distal end of the insertion assisting tool which is picked up by an image pickup section provided at a distal end of the inserting section and is displayed on an endoscope image display section.

31. The method for removing an endoscope inserting section according to claim 28, further comprising:
checking that the predetermined part of the inserting section is housed into the inner bore of the insertion assisting tool, by using an image of a distal end of the insertion assisting tool which is picked up by an image pickup section provided at a distal end of the inserting section.

32. The method for removing an endoscope inserting section according to claim 29, further comprising:
checking that the predetermined part of the inserting section is housed into the inner bore of the insertion assisting tool, by using an image of a distal end of the insertion assisting tool which is picked up by an image pickup section provided at a distal end of the inserting section and is displayed on an endoscope image display section.
